(19) United States
(12) Patent Application Publication
(10) Pub. No.: US 2007/0048748 A1
(43) Pub. Date: Mar. 1, 2007

(54) Mutant polymerases for sequencing and genotyping

(75) Inventors: John G.K. Williams, Lincoln, NE (US); Jon P. Anderson, Lincoln, NE (US); Teresa M. Urlacher, Wahoo, NE (US); David L. Steffens, Lincoln, NE (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LI-COR, INC., Lincoln, NE (US)

(21) Appl. No.: 11/234,677

(22) Filed: Sep. 23, 2005

Related U.S. Application Data
(60) Provisional application No. 60/626,552, filed on Nov. 10, 2004.
(60) Provisional application No. 60/613,560, filed on Sep. 24, 2004.

(51) Int. Cl. C12Q 1/68; C07H 21/04; C12P 21/06; C12N 9/22; C12N 15/74; C12N 1/21

(52) U.S. Cl. 435/6; 435/69.1; 435/199; 435/252.3; 435/471; 536/23.2

(57) ABSTRACT

The invention relates to the discovery of novel mutant DNA polymerases that possess altered kinetics for incorporating phosphate-labeled nucleotides during polymerization. The invention further relates to the use of these mutant DNA polymerases in sequencing and genotyping methods.

### INCORPORATION BY REFERENCE

This application claims the benefit of U.S. Patent Application No. 60/613,560, filed Sep. 24, 2004, entitled “Composition and Method for Nucleic Acid Sequencing,” and U.S. Patent Application No. 60/626,552, filed Nov. 10, 2004, both of which are incorporated by reference herein, in their entirety and for all purposes.

### GOVERNMENT RIGHTS

The invention described herein was made with support from U.S. government grants P01 HG003015-01 0003 and R44 HG002292-02. Accordingly, the U.S. government may have certain rights in the invention.

### FIELD OF THE INVENTION

The invention relates to the discovery of novel mutant DNA polymerases that possess altered kinetics for incorporating phosphate-labeled nucleotides during polymerization. The invention further relates to the use of these mutant DNA polymerases in sequencing and genotyping methods.

### BACKGROUND OF THE INVENTION

The primary sequences of nucleic acids are crucial for understanding the function and control of genes and for applying many of the basic techniques of molecular biology. In fact, rapid DNA sequencing has taken on a more central role after the goal to elucidate the entire human genome has been achieved. DNA sequencing is an important tool in genomic analysis as well as other applications, such as genetic identification, forensic analysis, genetic counseling, medical diagnostics, and the like. With respect to the area of medical diagnostic sequencing, disorders, susceptibilities to disorders, and prognoses of disease conditions can be correlated with the presence of particular DNA sequences, or the degree of variation (or mutation) in DNA sequences, at one or more genetic loci. Examples of such phenomena include human leukocyte antigen (HLA) typing, cystic fibrosis, tumor progression and heterogeneity, p53 proto-oncogene mutations, and ras proto-oncogene mutations (see, Gyllensten et al., PCR Methods and Applications, 1:91-98 (1991); U.S. Pat. No. 5,578,443, issued to Santamaria et al.; and U.S. Pat. No. 5,776,677, issued to Tsui et al.).

Various approaches to DNA sequencing exist. The dideoxy chain termination method serves as the basis for all currently available automated DNA sequencing machines, whereby labeled DNA elongation is randomly terminated within particular base groups through the incorporation of chain-terminating inhibitors (generally dideoxynucleoside triphosphates) and size-ordered by either slab gel electrophoresis or capillary electrophoresis (see, Sanger et al., Proc. Natl. Acad. Sci., 74:5463-5467 (1977); Church et al., Science, 240:185-188 (1988); Hunkapiller et al., Science, 254:59-67 (1991)). Other methods include the chemical degradation method (see, Maxam et al., Proc. Natl. Acad. Sci., 74:560-564 (1977), whole-genome approaches (see, Fleischmann et al., Science, 269:496 (1995)), expressed sequence tag sequencing (see, Velculescu et al., Science, 270 (1995)), array methods based on sequencing by hybridization (see, Koster et al., Nature Biotechnology, 14:1123 (1996)), highly parallel pyrosequencing, and single molecule sequencing (SMS) (see, Jett et al., J. Biomol. Struct. Dyn. 7:301 (1989); Schecker et al., Proc. SPIE-Int. Soc. Opt. Eng. 2386:4 (1995)).

There have been several improvements in the dideoxy chain termination method since it was first reported in the mid-1980s with enhancements in the areas of separating technologies (both in hardware formats & electrophoresis media), fluorescence dye chemistry, polymerase engineering, and applications software. The emphasis on sequencing the human genome with a greatly accelerated timetable along with the introduction of capillary electrophoresis instrumentation that permitted more automation with respect to the fragment separation process allowed the required scale-up to occur without undue pressure to increase laboratory staffing. However, despite such enhancements, the reductions in the cost of delivering finished base sequence have been marginal, at best.

In general, present approaches to improve DNA sequencing technology have either involved: (1) a continued emphasis to enhance throughput while reducing costs via the dideoxy chain termination method; or (2) a paradigm shift away from the dideoxy chain termination method such as sequencing by a non-electrophoretic method.

Although several non-electrophoretic DNA sequencing methods have been demonstrated or proposed, all are limited by short read lengths. For example, matrix-assisted laser desorption/ionization (MALDI) mass spectrometry, which separates DNA fragments by molecular weight, is only capable of determining about 50 nucleotides of DNA sequence due to fragmentation problems associated with ionization. Other non-electrophoretic sequencing methods depend on the cyclic addition of reagents to sequentially identify bases as they are either added or removed from the subject DNA. However, these procedures all suffer from the same problem as the classical Edman degradation method for protein sequencing, namely that synchronization among molecules decays with each cycle because of incomplete reaction at each step. As a result, current non-electrophoretic sequencing methods are unsuitable for sequencing longer portions of DNA.

The DNA polymerases employed in known sequencing methods are thermophilic or thermostable DNA polymerases such as Taq DNA polymerase derived from the bacterium Thermus aquaticus, Pfu DNA polymerase derived from the bacterium Pyrococcus furiosus, Tli DNA polymerase (also called Vent polymerase) derived from the bacterium Thermococcus litoralis and others. Thermostable DNA polymerases also play a crucial role in current methods of DNA amplification and sequencing. Some improvements in these methods have been made in recent years, particularly in DNA sequencing and the polymerase chain reaction. There are a number of mutants that have been generated, for example, DNA polymerase mutants that lack exonuclease activity (e.g., Vent_{R}® (exo-) DNA polymerase and Deep Vent_{R}™ (exo-) DNA polymerase from New England Biolabs; Therminator™ DNA polymerase from New England Biolabs; and KOD Hifi™ DNA polymerase from Novagen).

One of the most important characteristics of thermostable polymerases is their error rate. Error rates are measured using different assays, and as a result, estimates of error rates may vary, particularly from one laboratory to another. Polymerases lacking 3′→5′ exonuclease activity generally have higher error rates than polymerases with exonuclease activity. The total error rate of Taq polymerase has been variously reported between 1×10⁻⁴ to 2×10⁻⁵ errors per base pair. Pfu polymerase appears to have the lowest error rate at about 1.5×10⁻⁶ error per base pair, and Tli polymerase is known to be intermediate between Taq and Pfu. Although error rate is a significant factor when choosing a DNA polymerase, it is not the only factor. Reliability, stability and catalytic rate of the enzyme are equally important.

Clearly, there is an untapped potential in genetically modified DNA polymerases, which could provide significant advantages over their natural counterparts that are used today. There is indeed a need for more effective and efficient enzymes that can be used in methods of non-electrophoretic DNA sequencing. The present invention satisfies this and other needs.

### BRIEF SUMMARY OF THE INVENTION

The present invention provides novel mutant DNA polymerases that possess altered kinetics for incorporating phosphate-labeled nucleotides during polymerization. One major advantage of the mutant polymerases of the present invention is their faster incorporation kinetics for phosphate-labeled deoxynucleotide-triphosphates (dNTPs) during polymerization of DNA strands in comparison to native DNA polymerases. Another advantage of the present invention is that the mutant DNA polymerases reduce the cost of sequencing and genotyping due to their altered kinetics (e.g., faster kinetics). As such, the mutant DNA polymerases can be employed in various methods, including single-molecule DNA sequencing and genotyping methods.

In one embodiment, the present invention provides a mutant DNA polymerase, wherein the amino acid sequence of the phosphate region of said mutant DNA polymerase comprises two or more mutations not present in the phosphate region of the most closely related native DNA polymerase, and wherein said two or more phosphate region mutations increase the rate at which said mutant DNA polymerase incorporates phosphate-labeled nucleotides. In a related embodiment, the mutant DNA polymerase, or at least the phosphate region of said mutant polymerase, is derived from a Family A or Family B polymerase. In yet another related embodiment, the mutant DNA polymerase is a chimera combining homologous regions from distinct polymerases (as described, e.g., by Wang et al., J. Biological Chemistry, 270:26558-26564 (1995); Villbrandt et al., Protein Engineering, 13:645-654 (2000); Boudsocq et al., J. Biological Chemistry, 279:32932-32940 (2004)). For example, the phosphate region of one polymerase could be swapped for the phosphate region of another polymerase to create a new chimera.

In another embodiment, the invention provides a mutant 9°N DNA polymerase, wherein the amino acid sequence of the phosphate region of the 9°N DNA polymerase comprises two or more mutations not present in the phosphate region of native 9°N DNA polymerase, and wherein the two or more phosphate region mutations increase the rate at which said mutant DNA polymerase incorporates phosphate-labeled nucleotides. In a related embodiment, the mutant 9°N DNA polymerase incorporates phosphate-labeled nucleotides at an increased rate relative to 9°N-A485L DNA polymerase (SEQ ID NO: 2), comprises an alanine to leucine mutation at amino acid position 485, and further comprises one or more additional mutations in its phosphate region. In yet another related embodiment, the one or more additional mutations are selected from the group consisting of a mutation at amino acid position 352, 355, 408, 460, 461, 464, 480, 483, 484, and 497, and combinations thereof. In another related embodiment, the mutant 9°N DNA polymerase comprises a mutation at amino acid position 484 as one of the additional mutations. In yet another related embodiment, the additional mutations include mutations at amino acid positions 408, 464, and 484. In some embodiments of the mutant 9°N DNA polymerase of the invention, the mutation at position 408 is selected from the group consisting of tryptophan, glutamine, histidine glutamic acid, methionine, asparagine, lysine, and alanine; the mutation at position 464 is selected from the group consisting of glutamic acid and proline; and the mutation at position 485 is tryptophan. In yet another related embodiment, the amino acids at positions 408, 464, and 484 in the mutant 9°N DNA polymerase are tryptophan, glutamic acid, and tryptophan, respectively.

In another embodiment, the invention provides a mutant DNA polymerase comprising an amino acid sequence region homologous to amino acids 325 to 340 of SEQ ID NO:2, wherein the region contains at least one mutation and wherein the mutant DNA polymerase incorporates phosphate-labeled nucleotides at an increased rate relative to a 9N-A485L DNA polymerase (SEQ ID NO:2). In a preferred embodiment, the at least one mutation is at an amino acid position selected from the group consisting of amino acid positions 329, 332, 333, 336 and 338. In another preferred embodiment, the mutant DNA polymerase comprises an insertion or a deletion of at least 1 amino acid in an amino acid sequence region homologous to amino acids 325 to 340 of SEQ ID NO:2. In a related embodiment, the at least one mutation is an insertion or a deletion of at least 10 amino acids. In yet another embodiment, the at least one mutation is an insertion of amino acids REAQLSEFFPT at position 329.

In yet another embodiment, the invention provides a mutant DNA polymerase comprising an amino acid sequence region homologous to amino acids 473 to 496 of SEQ ID NO: 2, wherein the region contains at least one mutation and wherein the mutant DNA polymerase incorporates phosphate-labeled nucleotides at an increased rate relative to 9N-A485L DNA polymerase (SEQ ID NO: 2). In a related embodiment, the at least one mutation is at an amino acid position selected from the group consisting of amino acid positions 480, 483, 484 and 485. In another preferred embodiment, the mutant DNA polymerase comprises an insertion or a deletion of at least 1 amino acid in an amino acid sequence region homologous to amino acids 473 to 496 of SEQ ID NO:2. In a related embodiment, the at least one mutation is an insertion or a deletion of at least 10 amino acids. In yet another embodiment, the at least one mutation in the DNA polymerase is an insertion at a position corresponding to position 485 in SEQ ID NO:2 of an amino acid sequence selected from the group consisting of PIKILANSYRQRW, TIKILANSYRQRQ and PIKILANLDYRQRL. In yet another embodiment, the mutant DNA polymerase comprises the mutated sequence of amino acids found at region 473 to 496 in any of the DNA polymerase sequences set forth in SEQ ID NO: 4 through SEQ ID NO: 750, and wherein the mutant DNA polymerase comprises the mutated sequence at a region which is homologous to region 473 to 496 in SEQ ID NO: 2.

In another embodiment, the invention provides a mutant DNA polymerase, wherein the mutant DNA polymerase incorporates phosphate-labeled nucleotides at an increased rate relative to 9N-A485L DNA polymerase (SEQ ID NO: 2), and comprises (i) a first amino acid sequence region homologous to amino acids 325 to 340 of SEQ ID NO:2, wherein this first region contains at least one mutation; and (ii) a second amino acid sequence region homologous to amino acids 473-496 of SEQ ID NO:2, wherein this second region contains at least one mutation. In a related embodiment, the at least one mutation in the first region is at an amino acid position selected from the group consisting of amino acid positions 329, 332, 333, 336 and 338, and the at least one mutation in the second region is at an amino acid position selected from the group consisting of amino acid positions 480, 483, 484 and 485. In certain embodiments, the mutations include insertions or deletions of one or more amino acids in the two regions, including insertions or deletions of up to ten or more amino acids. In one embodiment, the mutation in the first region is an insertion of amino acids REAQLSEFFPT at the position corresponding to position 329 in SEQ ID NO: 2 and the mutation in the second region is an insertion of PIKILANSYRQRW at the position corresponding to position 485 in SEQ ID NO: 2. In yet another embodiment, the first region in the mutant polymerase comprises the mutated sequence of amino acids found at region 325 to 340 in any of the DNA polymerase sequences set forth in SEQ ID NO: 4 through SEQ ID NO: 750, and the second region comprises the mutated sequence of amino acids found at region 473 to 496 in any of the DNA polymerase sequences set forth in SEQ ID NO: 4 through SEQ ID NO: 750.

The invention also provides a mutant 9°N DNA polymerase comprising at least two mutations in the phosphate region, including an A485L mutation, wherein the mutant 9°N DNA polymerase incorporates phosphate-labeled nucleotides at an increased rate relative to 9°N-A485L DNA polymerase (SEQ ID NO: 2), and wherein the increased rate is at least three times, at least seven times, at least twenty times, or at least fifty times faster than that catalyzed by 9°N-A485L DNA polymerase, as based on primer extension assays analyzed on polyacrylamide gels.

In yet another embodiment, the invention provides the mutant 9°N DNA polymerase of SEQ ID NO. 568. In a related embodiment, the invention provides the mutant 9°N DNA polymerase of SEQ ID NO. 568, where the mutant sequence further comprises one or more additional mutations and wherein the one or more additional mutations are selected from the group consisting of an alteration in amino acid identity, an insertion of one or more amino acids, and the deletion of one or more amino acids. In yet another related embodiment, the mutant 9°N DNA polymerase includes one mutation relative to SEQ ID NO. 568, wherein said additional mutation is in the phosphate region of the mutant 9°N DNA polymerase. In a related embodiment, the additionally mutated amino acid is selected from the group consisting of the asparagine at position 491 and lysine at position 487.

In another embodiment, the invention provides a mutant 9°N DNA polymerase of SEQ ID NO. 568 and conservative modifications thereof. Examples of conservative amino acid mutations are well-known in the art and described, e.g., in U.S. patent set forth, for instance, in U.S. Pat. No. 5,364,934. In a related embodiment, the conservative mutations lie outside the phosphate region of said mutant polymerases.

The invention also provides a mutant 9°N DNA polymerase with an amino acid sequence selected from the group consisting of the even-numbered SEQ ID NOs 4 through 750. In a related embodiment, the invention provides a purified nucleic acid sequence encoding the amino sequence of any of the even-numbered SEQ ID NOs: 4 through 750. In a related embodiment, the invention provides a purified nucleic acid sequence encoding a polymerase represented by the group consisting of the even-numbered SEQ ID NOs: 4 through 750. In a related embodiment, the invention provides the nucleic acids of the odd-numbered SEQ ID NOs: 3-749.

The invention also provides a method for identifying polymerases with improved suitability for a nucleotide sequencing process, wherein the improved suitability is measured relative to that of a parent polymerase, comprising: (1) assaying the rate of phosphate-labeled nucleotide incorporation by a test mutant polymerase, wherein said phosphate region of said test polymerase is at least 90% identical to said parent polymerase; (2) determining if said rate of phosphate-labeled nucleotide incorporation by said test mutant polymerase is suitable for said nucleotide sequencing process; and, if said rate of phosphate-labeled nucleotide incorporation is suitable, then identifying the test mutant polymerase as such. In a related embodiment, the method includes an additional step, wherein if said rate of phosphate-labeled nucleotide incorporation is not suitable, steps (1) and (2) are repeated with a second test mutant polymerase until a suitable polymerase is identified. In yet another related embodiment, if said second test mutant comprises each of the mutations in the previous test mutant polymerase, and further comprises at least one additional mutation relative to the previous test mutant polymerase.

In a related embodiment of the method for identifying suitable polymerases, the parent polymerase is a thermostable polymerase. In yet another related embodiment, the amino acid sequence of said parent polymerase is at least 90% identical to the amino acid sequence of 9°N-A485L DNA polymerase (SEQ ID NO: 2). In yet another related embodiment, the amino acid sequence of said parent polymerase is at least 95% identical to the amino acid sequence of 9°N-A485L DNA polymerase (SEQ ID NO: 2). In yet another related embodiment, the improved polymerase is a polymerase which incorporates between 1 and 20 phosphate-labeled nucleotides per second or, preferably, between 5 and 15 phosphate-labeled nucleotides per second. In yet another embodiment of the method, the sequencing process suitable for the improved polymerase is a field-switch polynucleotide sequencing process.

In another embodiment, the invention provides a mutant DNA polymerase, wherein the amino acid sequence of the phosphate region of the mutant DNA polymerase comprises one or more mutations not present in the phosphate region of the most closely related native DNA polymerase, and wherein the one or more phosphate region mutations increase the rate at which the mutant DNA polymerase incorporates a phosphate-labeled nucleotide. In a related embodiment, the mutant DNA polymerase is a Family A DNA polymerase. In yet another related embodiment, the mutant DNA polymerase is a mutant Klenow DNA polymerase.

In another related embodiment, the invention provides a mutant Klenow polymerase which incorporates phosphate-labeled nucleotides at an increased rate relative to the Klenow DNA polymerase of SEQ ID NO: 752, wherein the mutant Klenow DNA polymerase comprises one or more phosphate region mutations. In a related embodiment, the additional mutations are selected from the group consisting of a mutation at amino acid position 423 and 504, and combinations thereof. In yet another related embodiment of the mutant Klenow DNA polymerase, the amino acid at position 423 is mutated or, alternately, the amino acid at position 504 is mutated. In certain related embodiments, the amino acid at position 423 is lysine or glutamic acid, and the amino acid at position 504 is glycine. In yet another related embodiment, the mutant Klenow DNA polymerase incorporates phosphate-labeled nucleotides at a rate at least three times faster than the Klenow polymerase of SEQ ID NO: 752. In yet another related embodiment, the invention provides the mutant Klenow DNA polymerases of SEQ ID NO: 756, 758, or 764, as well as polymerases with conservative mutations or mutations which do not substantially alter the rate at which the mutant polymerase incorporates phosphate-labeled nucleotides. In yet another related embodiment, the invention provides purified nucleic acids encoding the mutant Klenow DNA polymerases of the invention.

In another embodiment, the invention provides a mutant DNA polymerase, wherein the amino acid sequence of the phosphate region of the mutant DNA polymerase comprises one or more mutations not present in the phosphate region of the most closely related native DNA polymerase, and wherein the one or more phosphate region mutations increase the rate at which the mutant DNA polymerase incorporates a phosphate-labeled nucleotide, wherein the mutant DNA polymerase is a mutant Taq DNA polymerase. In a related embodiment, the mutant Taq DNA polymerase incorporates phosphate-labeled nucleotides at an increased rate relative to the Taq DNA polymerase of SEQ ID NO: 766.

In a related embodiment, the mutant Taq mutations are selected from the group consisting of a mutation at amino acid positions 589, 617, 645, 691, 673, and 726, and combinations thereof. In a related embodiment, the amino acid at position 617 is isoleucine. In yet another related embodiment, the mutated amino acid at position 645 is selected from the group consisting of histidine, phenylalanine, lysine and tryptophan. In yet another releated embodiment, the amino acid at position 691 is tyrosine. In yet another related embodiment, the amino acid at position 693 is glycine. In yet another related embodiment, the amino acid at position 726 is serine. In yet another related embodiment, the amino acid at position 589 is aspartic acid and the amino acid at position 645 is histidine. In yet another related embodiment, the mutant Taq DNA polymerase of the invention incorporates phosphate-labeled nucleotides at a rate at least two times faster, or between five and fifteen times faster, than the Taq polymerase of SEQ ID NO: 766. In yet another embodiment, the invention provides the mutant Taq DNA polymerase of SEQ ID NO: 768, 770, 772, 774, 776, 778, 780, 782 or 784, as well as derivatives of these mutant Taq DNA polymerases with additional conservative mutations or mutations which do not substantially alter the rate at which the mutant polymerase incorporates phosphate-labeled nucleotides. In yet another related embodiment, the invention provides purified nucleic acids encoding the mutant Taq DNA polymerases of the invention.

The invention additionally provides a mutant DNA polymerase selected from the group consisting of the mutant DNA polymerases represented by the even-numbered sequences of SEQ ID NOs: 4-750, 754-764, and 768-784, as well as a mutant DNA polymerase wherein the phosphate region of said mutant DNA polymerase is identical to the phosphate region of a polymerase selected from the group consisting of the mutant DNA polymerases represented by the even-numbered sequences of SEQ ID NOs: 4-750, 754-764, and 768-784. In a related embodiment, the invention provides a mutant DNA polymerase selected from the group consisting of the mutant DNA polymerases represented by the even-numbered sequences of SEQ ID NOs: 4-750, wherein the mutant DNA polymerase incorporates phosphate-labeled nucleotides at an increased rate relative to the DNA polymerase of SEQ ID NO: 2. In another related embodiment, the invention provides a mutant DNA polymerase selected from the group consisting of the mutant DNA polymerases represented by the even-numbered sequences of SEQ ID NOs: 754-764, wherein the mutant DNA polymerase incorporates phosphate-labeled nucleotides at an increased rate relative to the DNA polymerase of SEQ ID NO: 752. In yet another related embodiment, the invention provides a mutant DNA polymerase selected from the group consisting of the mutant DNA polymerases represented by the even-numbered sequences of SEQ ID NOs: 768-784, wherein said mutant DNA polymerase incorporates phosphate-labeled nucleotides at an increased rate relative to the DNA polymerase of SEQ ID NO: 766.

In a preferred embodiment, the phosphate-labeled nucleotides incorporated by the mutant DNA polymerases of the invention are γ-phosphate-labeled nucleotides. In one embodiment, the polymerase incorporates phosphate-labeled nucleotides in which the label is a moiety capable of complexing with DNA. The DNA-complexing moiety may include intercalating dyes (e.g., FIG. 6), major-groove binders, minor-groove binders and moieties capable of covalent crosslinking to DNA. In yet another embodiment, the polymerase incorporates phosphate-labeled nucleotides where the label is a single or double-stranded oligonucleotide, i.e., an oligoLabel. In one aspect, the oligoLabel is attached to the gamma phosphate of the nucleotide triphosphate through a linker. In related embodiments, the linker may be attached to the oligoLabel by non-covalent bonds (e.g., hydrophobic or electrostatic associations, as depicted in FIG. 7) or covalent bonds (e.g., an amide bond, as depicted in FIG. 8).

In yet another embodiment, the invention provides mutant DNA polymerases which substantially lack exonuclease activity. In still another embodiment, the mutant DNA polymerases provided by the invention are derived from a family B polymerase. In yet another embodiment, the mutant DNA polymerases provided by the invention are derived from a family A polymerase. In a preferred embodiment, the amino acid sequence of the mutant DNA polymerase is derived from the amino acid sequence of a polymerase selected from the group consisting of a 9°N DNA polymerase derived from Thermococcus species 9°N-7; a Tli DNA polymerase derived from Thermococcus litoralis; a DNA polymerase derived from Pyrococcus species GB-D; a KOD1 DNA polymerase derived from Thermococcus kodakaraensis; a Taq DNA polymerase derived from Thermus aquaticus; a Phi-29 polymerase derived from Bacillus subtilis phage phi-29; and a polymerase I Klenow fragment derived by proteolysis from the bacterium Escherichia coli (Henningsen, K., PNAS, 65:168 (1970); Brutlag et al., BBRC, 37:982 (1969); Setlow et al., JBC 247:224 (1972); Setlow, P. and Kornberg, A., JBC, 247:232 (1972)). The sequences of these native DNA polymerases are set forth in Table 6.

In another embodiment of the invention, any of the mutant DNA polymerases set forth in SEQ ID NO: 4 through SEQ ID NO: 750 (9°N mutants), SEQ ID 754-764 (Klenow mutants) or SEQ ID NO: 767 through SEQ ID NO: 784 (Taq mutants) can be used for DNA sequencing and/or genotyping. DNA sequencing methods include, but are not limited to, single-molecule sequencing, such as field-switch sequencing, charge-switch sequencing and/or electrokinetic sequencing. See, e.g., U.S. Pat. Nos. 6,232,075; 6,306,607; and 6,762,048; see also U.S. Pat. Nos. 6,936,702 and 6,869,764. Preferably, the mutant DNA polymerases are used in single-molecule sequencing or single-molecule genotyping. In another preferred embodiment, the mutant polymerases selected are those which exhibit increased rates of incorporation of phosphate-labeled nucleotides relative to, e.g., the parent polymerases whose amino sequences are provided by SEQ ID NO: 2, SEQ ID NO: 752 or SEQ ID NO: 756.

The instant invention also provides improved sequencing and genotyping methods that employ the mutant DNA polymerases. Particularly, the invention contemplates a method of DNA sequencing, wherein the method comprises (i) immobilizing at least one complex comprising a target nucleic acid, a primer nucleic acid, and a mutant DNA polymerase onto a surface; (ii) contacting the surface with a plurality of charged particles comprising at least one type of phosphate-labeled nucleotide triphosphate (NTP) (e.g., γ-phosphate-labeled NTP) by applying an electric field; (iii) reversing the electric field to transport unbound charged particles away from the surface; and (iv) detecting the incorporation of a phosphate-labeled NTP into a single molecule of the primer nucleic acid. The mutant DNA polymerase employed in this method can preferably be any of the mutant DNA polymerases represented by the even-numbered sequences of SEQ ID NOs: 4-750, 754-764, and 768-784. The phosphate-labeled NTPs can be further labeled with polyethylene glycol (PEG). The incorporation of an NTP can be detected by a total internal reflection fluorescent microscope or other detection devices.

The method of DNA sequencing can employ immobilizing at least one complex including a target nucleic acid, a primer nucleic acid, and a mutant DNA polymerase onto a surface that is an indium-tin oxide (ITO) electrode coated by a permeation layer. Complexes can be immobilized onto the surface by covalent bonding, non-covalent bonding, ionic bonding or the like. The method of DNA sequencing can employ contacting the surface with a plurality of charged particles. The charged particles include, but are not limited to, nanoparticles, charged polymers (e.g., DNA), and combinations thereof. The charged particles can further comprise at least one dye. In addition, the nanoparticles can be silica-DNA nanoparticles. For example, electrokinetic DNA sequencing can be performed in a two-electrode chamber such as a microtiter plate fitted with two electrodes. One advantage of this method is that over two-hundred different single DNA molecules can be sequenced simultaneously in a single well at a maximum rate of about 10 to about 200 nucleotides per second per molecule and at read lengths of 20 kilobases (kb) or more. Another advantage of this method is the lower cost of sequencing as compared to other long read approaches due to the high degree of multiplexing and the substitution of microtiter plates for expensive micro- or nano-fabricated devices.

In an additional embodiment, the invention provides mutant polymerases wherein the mutant polymerases have one, two, or more anchor sequences for immobilizing the polymerases on a solid surface and/or associating the polymerase with a target nucleic acid, in order to increase the processivity index of the polymerase. DNA polymerases comprising such anchor sequences are described, e.g., in U.S. patent application Ser. No. 10/821,689 (published as 2005/0042633A1), incorporated herein by reference.

The invention further encompasses methods of DNA genotyping. Such methods can employ genotyping by sequencing specific DNA segments from the target genome or randomly-selected DNA segments from the target genome to identify a subset of the genetic variation. Alternatively, efficient sequencing via the methods of the present invention can provide information about a complete genotype (i.e., by sequencing the entire genome). Sequence analysis performed using the polymerases and/or methods of the present invention can provide reads up to 20 kilobases and longer. Such long reads, each originating from a single DNA molecule, allow determination of haplotypes and long-range genomic rearrangements that are generally difficult to obtain with known sequencing and genotyping methods.

Other objects, features, and advantages of the present invention will be apparent to one of skill in the art from the following detailed description and figures.

### BRIEF DESCRIPTION OF THE DRAWINGS

The present invention is best understood when read in conjunction with the accompanying figures which serve to illustrate the various embodiments. It is understood, however, that the invention is not limited to the specific embodiments disclosed in the figures.

FIG. 1 shows an alignment of amino acid sequences of five Family B DNA polymerases. Residues conserved between the various polymerases are shown in bold. Abbreviations: 9N_pol: 9°N DNA polymerase; Kod1_pol: DNA polymerase from Thermococcus kodakaraensis; PWO: DNA polymerase from Pyrococcus woesei; Pfu: DNA polymerase from bacterium Pyrococcus furiosus; Vent: Thermococcus litoralis DNA polymerase.

FIG. 2 shows a nucleotide configuration in a method of the present invention in which dNTPs are attached to a nanoparticle by a linker to the γ-phosphate group. This sort of nucleotide is included within the definition of the term “phosphate-labeled nucleotide,” a substrate of the mutant DNA polymerases described herein.

FIG. 3 shows an electrokinetic cycle in an electrokinetic sequencing method that employs the mutant DNA polymerases. FIG. 3A illustrates the accumulation of negatively-charged particles above immobilized polymerase-DNA complexes on a positively-charged indium-tin oxide (ITO) electrode. FIG. 3B illustrates the movement of unbound particles away from the ITO electrode when the electric field is reversed. The ITO electrode surface is illuminated by total internal reflection (arrows) and the particles retained by the polymerase-DNA complexes are imaged.

FIG. 4 (left) shows a diagram of a circular template that is permanently associated with an anchored mutant DNA polymerase of the present invention, while still being able to slide through the DNA binding groove to permit primer extension. The tunnel formed by polymerase immobilization is roughly the same dimension as a DNA sliding clamp. FIG. 4 (right) shows the crystal structure of Therminator™ polymerase with 6×His engineered loops inserted at positions K53 and K229. The open/closed conformational change involves movement of the helices O and N as shown to admit a nucleotide to the binding pocket. DNA (“ssDNA template”) in the DNA binding cleft is also shown.

FIG. 5 depicts the structure of dUTP-PEG8-P2-AlexaFluor633 (a γ-labeled NTP), a nucleotide triphosphate attached to a dye and linker by a nitrogen-phosphorous bond.

FIG. 6 depicts a terminal phosphate-labeled nucleotide in which the label is an intercalating dye, JOJO-1, capable of complexing with DNA.

FIG. 7 depicts an oligoLabel joined to a nucleotide, wherein the oligoLabel is attached to the nucleotide (dCTP) via non-covalent interactions with JOJO-1 and a linker.

FIG. 8 depicts a covalent crosslinked complex between psoralen and an oligoLabel.

FIG. 9 depicts steps in the enzymatic pathway of DNA replication by polymerases. The action of a variety of DNA polymerases can be defined by a reaction pathway that describes the steps involved in the process of DNA replication. This pathway is typically presented in six steps as shown in FIG. 9 (see, e.g., Joyce, C. M. and Benkovic, S. J., Biochemistry, 43:14317-14324 (2004)). Step 1, binding of DNA by the polymerase; Step 2, binding of dNTP by the polymerase-DNA complex; Step 3, rearrangement of secondary structure elements from “open” (E_{O}) to “closed” (E_{C}) conformation (seen in most polymerases, but not all), followed by additional unspecified conformational changes and binding of Mg²⁺ ions to form the active site (Steps 3.1 and 3.2); Step 4, phosphoryl transfer attaching the nucleotide to the DNA; Step 5, reversal of earlier conformational changes to restore the open conformation of the enzyme; and Step 6, release of pyrophosphate (PPi). In most polymerases studied, the rate-limiting step occurs between Steps 3 and 4 (Shah et al., J. Biol. Chem., 276:10824-10831 (2001); Arndt et al., Biochemistry, 40:5368-5375 (2001); Purohit et al., Biochemistry, 42:10200-10211 (2003); Fidalgo da Silva et al., J. Biol. Chem., 277:40640-40649 (2002); Rothwell et al., Molecular Cell, 19:345-355 (2005); Yang et al., Biophysical Journal, 86:3392-3408 (2004)).

FIG. 10 shows the results of a gel extension assay using saturating amounts of the selected purified polymerases.

FIG. 11 shows the analysis of the assay described in FIG. 10, including the average rate (nucleotides per second) for each of the indicated enzymes.

FIG. 12 shows steps in the identification of the phosphate region of 9°N DNA polymerase (see Example 3). FIG. 12a shows 9°N DNA polymerase holoenzyme (1QHT.pdb) superposed with DNA and TTP from RB69 polymerase (1IG9.pdb). FIG. 12b shows amino acids selected from FIG. 12a by proximity to dTTP (within 15 Å) and constrained by location between the gamma-phosphate of the dTTP and the enzyme surface. FIG. 12c shows the secondary structural elements in 9N DNA polymerase containing amino acids identified in FIG. 12b.

### DETAILED DESCRIPTION OF THE INVENTION

### I. Definitions

The following definitions are set forth to illustrate and define the meaning and scope of the various terms used to describe the invention herein. As such, the following terms have the meanings ascribed to them unless specified otherwise.

A “native DNA polymerase,” as used herein, is used to describe DNA polymerases that have not previously been genetically altered or modified as described herein. Examples of native DNA polymerases include, but are not limited to, a 9°N DNA polymerase derived from Thermococcus species 9°N-7; a Tli DNA polymerase derived from Thermococcus litoralis; a DNA polymerase derived from Pyrococcus species GB-D; a KOD1 DNA polymerase derived from Thermococcus kodakaraensis; a native Taq DNA polymerase derived from Thermus aquaticus; a native Phi-29 polymerase derived from Bacillus subtilis phage phi-29; and a polymerase I Klenow fragment derived from the bacterium Escherichia coli. Sequences of representative native DNA polymerase sequences are provided in Table 6. Native DNA polymerases may be used as parent polymerases in methods of the invention which relate to the identification of mutant polymerases, derived from the parent polymerases, which exhibit altered and typically improved kinetics of incorporating phosphate-labeled nucleotides.

The term “mutant DNA polymerase” refers to any DNA polymerase that has been genetically altered such that it contains one or more mutation (e.g., point mutation(s), deletion(s), insertion(s) and the like) in its polypeptide sequence compared to a native DNA polymerase of the same species.

The term “altered kinetics” means that the rate of polymerization (i.e., the incorporation of nucleotides into a DNA strand) of a DNA polymerase has been changed (e.g., increased or decreased) as compared to the rate of polymerization displayed by a naturally occurring or native DNA polymerase, and includes effects on the reaction mechanism that impact nucleotide binding and incorporation of the nucleotide.

The term “homologous position” means, for the purpose of the specification and claims, an amino acid position in a genetically altered polypeptide sequence of a specific protein (e.g., a mutant DNA polymerase) that corresponds, or is similar in position or structure, to the amino acid position of the naturally occurring or native polypeptide sequence of the specific protein (e.g., a native DNA polymerase). For example, the genetically altered polypeptide sequence of a mutant DNA polymerase can exhibit a point mutation at amino acid position N, such that the amino acid at position N is changed from, e.g., alanine in the native polymerase sequence, to leucine in the mutant polymerase sequence. Amino acid positions identified within this document are numbered according to the reference sequence for each polymerase type unless otherwise noted, with mutants of 9°N DNA polymerase numbered according to amino acid positions of SEQ ID NO. 2, mutants of Klenow DNA polymerase according to amino acid positions of SEQ ID NO. 752, and mutants of Taq DNA polymerase according to amino acid positions of SEQ ID NO. 766. A more specific example would be the phosphate regions of DNA polymerases which, when mutated, result in altered or improved kinetics of nucleotide incorporation. The phosphate region of a DNA polymerase is described in more detail below and in the Examples provided herein. Other specific homologous positions in 9N-A485L polymerase, Taq polymerase and Klenow polymerase are shown in Table 4, below.

TABLE 4
Homologous Residues in Various DNA Polymerases

| 9N-A485L | Taq | Klenow |
|---|---|---|
| SEQ ID NO. 2 | SEQ ID NO. 766 | SEQ ID NO. 752 |
| R359 | R726 | L504 |
| L408 | I645 | I423 |
| R484 | R691 | R469 |
| A485 | A693 | A471 |
| E598 | V617 | V395 |

The term “oligonucleotide” as used herein includes oligomers of nucleotides or analogs thereof, including deoxyribonucleosides, ribonucleosides, and the like. Typically, oligonucleotides range in size from a few monomeric units, e.g., 3-4, to several hundreds of monomeric units. Whenever an oligonucleotide is represented by a sequence of letters, it will be understood that the nucleotides are in 5′-3′ order from left to right and that “A” denotes deoxyadenosine, “C” denotes deoxycytidine, “G” denotes deoxyguanosine, “T” denotes thymidine, and “U” denotes deoxyuridine unless otherwise noted.

The term “nucleotide” as used herein refers to a phosphate ester of a nucleoside, e.g., mono-, di-, tri-, tetra-, penta-, polyphosphate esters, wherein the most common site of esterification is the hydroxyl group attached to the C-5 position of the pentose. Nucleosides also include, but are not limited to, synthetic nucleosides having modified base moieties and/or modified sugar moieties, e.g., described generally by Scheit, Nucleotide Analogs, John Wiley, N.Y. (1980). Suitable NTPs include both naturally occurring and synthetic nucleotide triphosphates, and are not limited to, ATP, dATP, CTP, dCTP, GTP, dGTP, TTP, dTTP, UTP, and dUTP. Preferably, the nucleotide triphosphates used in the methods of the present invention are selected from the group consisting of dATP, dCTP, dGTP, dTTP, dUTP, and combinations thereof. Preferably, nucleotide triphosphates are used, however, other phosphates such as mono-, di-, tetra-, penta-, and polyphosphate esters can also be used.

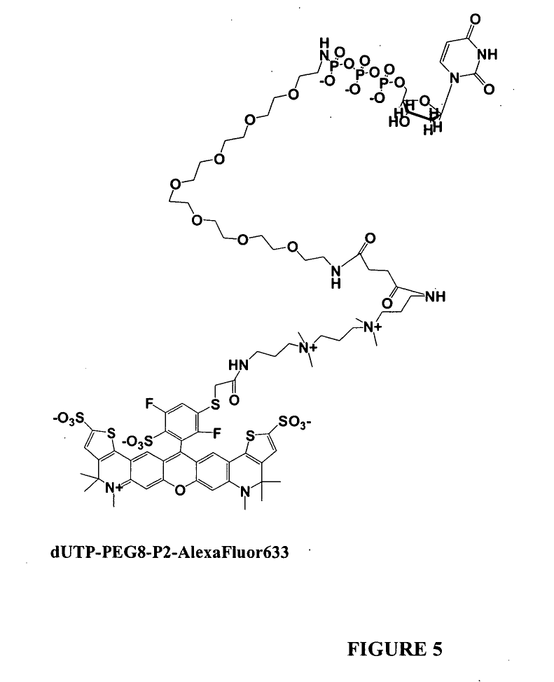

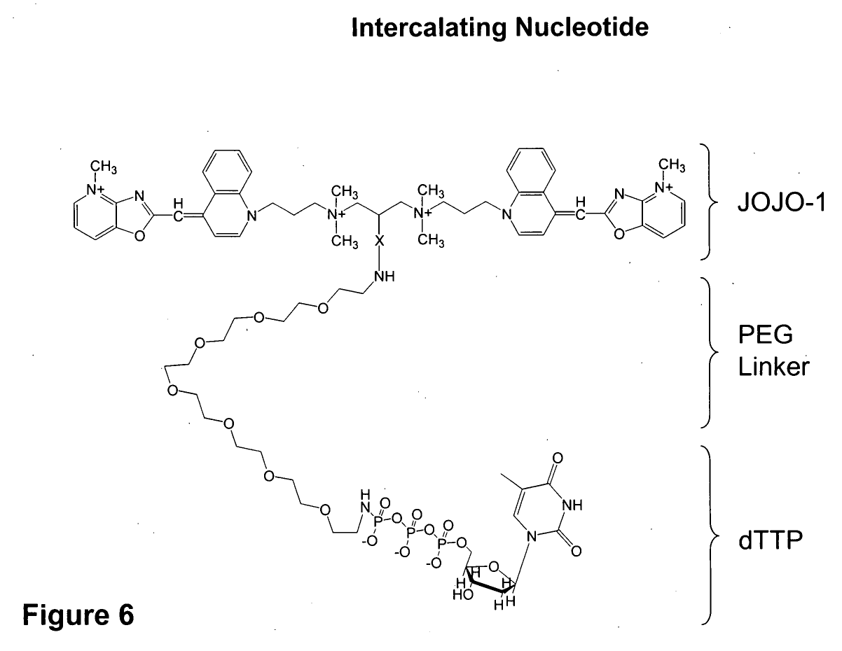

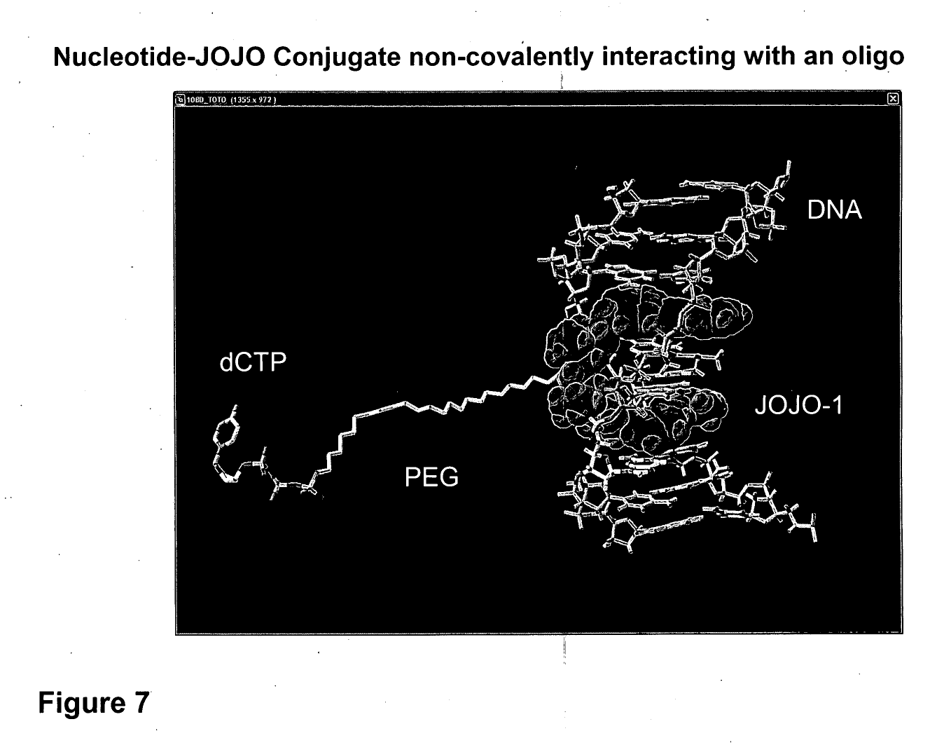

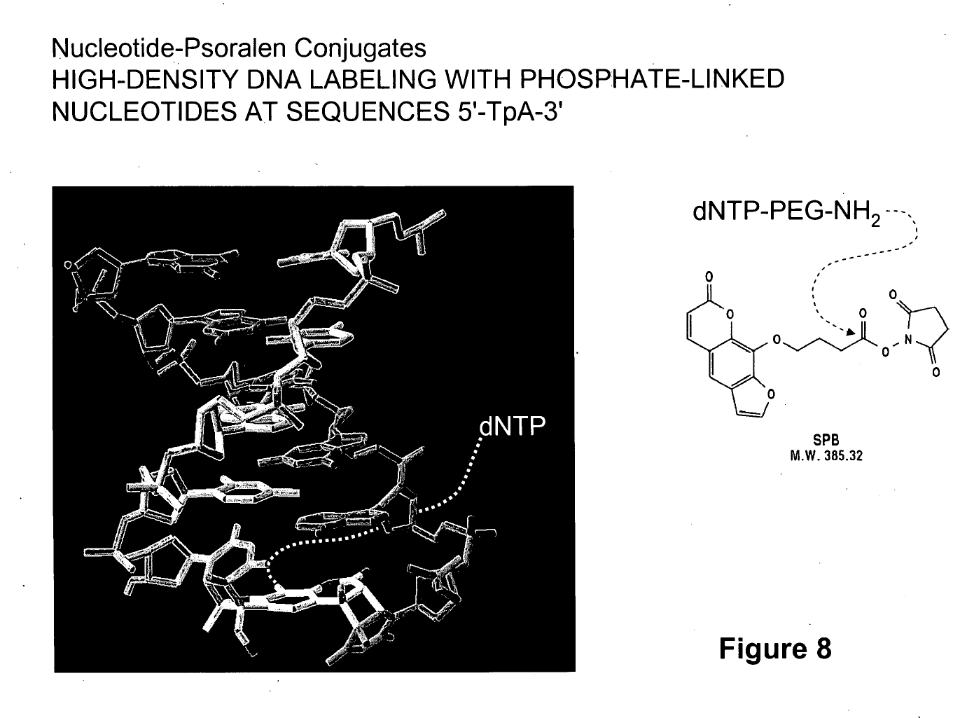

The terms “phosphate-labeled nucleotide triphosphate (NTP)” and “phosphate-labeled deoxynucleotide-triphosphate (dNTP)” are used interchangeably herein, and refer to any nucleotide (i.e., natural or synthetic) that contains a detectable label on any of its phosphate positions. The label, e.g., a dye, can be attached to the dNTP by a linker. The label or linker can be attached to the phosphate atom by a phosphorus-oxygen, phosphorus-nitrogen, phosphorus-sulfur, or phosphorus-carbon bond, for example, dUTP-PEG8-P2-AlexaFluor633 (see FIG. 5). The dNTP can also incorporate a polyethylene glycol (PEG) in addition to a dye label. For example, the dNTP can be a PEG-modified dNTP (e.g., dNTP with a PEG linker) with or without a dye label. One example of a phosphate-labeled nucleotide is a γ-labeled nucleotide. The term “γ-labeled” refers to a detectable label or an undetectable linker attached to any of the 3 phosphates on the nucleotide. The terms “γ-phosphate-labeled nucleotide triphosphate (NTP)” or “γ-phosphate-labeled deoxynucleotide-triphosphate (dNTP)” refer to any nucleotide (i.e., natural or synthetic) that contains a detectable label on its terminal, e.g., γ-phosphate, position. Preferably, nucleotide triphosphates are used, however other phosphates such as mono-, di-, tri, tetra-, penta-, and polyphosphate esters can also be used, wherein the label is preferably attached to the terminal phosphate, but may be attached to non-terminal phosphates. Certain labeled nucleotides suitable for use in the present invention include, but are not limited to, labeled nucleotides disclosed in for example, U.S. Pat. Nos. 6,232,075, 6,306,607, 6,936,702, 6,869,764, U.S. Patent Publication No. US2005/0042633, U.S. patent application Ser. No. 11/118,031, filed Apr. 29, 2005, Ser. No. 11/154,419, filed Jun. 15, 2005 and 60/648,091, filed Jan. 28, 2005. All of the foregoing patent publications and applications are incorporated herein by reference in their entirety.

The term “primer nucleic acid” refers to a linear oligonucleotide, which specifically anneals to a unique target nucleic acid sequence and allows for synthesis of the complement of the target nucleic acid sequence.

The phrase “target nucleic acid” refers to a nucleic acid or polynucleotide whose sequence identity or ordering or location of nucleosides is to be determined using the methods described herein.

The phrase “sequencing a nucleic acid,” in reference to a target nucleic acid, includes determination of partial as well as full sequence information of the target nucleic acid. That is, the term includes sequence comparisons, fingerprinting, and like levels of information about a target nucleic acid, as well as the express identification and ordering of nucleosides, usually each nucleoside, in a target nucleic acid. The term also includes the determination of the identification, ordering, and locations of one, two, or three of the four types of nucleotides within a target nucleic acid.

### II. Phosphate Region

DNA polymerases are classified into 6 families: A, B, C, X, Y and RT (Braithwaite et al., Nucleic Acids Res, 21:787 (1993); Delbos et al., J Exp Med, 201:1191 (2005); Hubscher et al., Annu Rev Biochem, 71:133 (2002); Ito and Braithwaite, Nucleic Acids Res, 19:4045 (1991); L. S. Kaguni, Annu Rev Biochem, 73:293 (2004); Southworth et al., Proc Natl Acad Sci USA, 93:5281 (1996); T. A. Steitz, J Biol Chem, 274:17395 (1999)). Family A polymerases perform both replication and repair functions, and include enzymes like Taq DNA polymerase, E. coli DNA polymerase I, T7 DNA polymerase and mitochondrial polymerase gamma. Family B polymerases include replicases such as the archaeal polymerase 9°N, the bacteriophage polymerases RB69 and phi-29, and the eukaryotic replicases alpha, delta and epsilon. Family C polymerases include bacterial replicases such as E. coli DNA polymerase III. Family X polymerases, involved in error-prone repair, include polymerases beta, lambda, mu, DP04 and terminal transferases. Finally, Family Y polymerases are the so-called lesion-bypass polymerases; they include polymerases eta, kappa, iota, and zeta. Polymerases within each family are structurally related. Structural models have been determined by x-ray crystallography for members of nearly every family.

The term “phosphate region” as used herein refers to a collection of secondary structure elements (i.e., helix, strand, coil) in a DNA polymerase which form a protein channel connecting, e.g., the γ-phosphate of a bound dNTP to the enzyme surface. As such, this channel would be occupied by a linker attached to the dNTP gamma-phosphate extending toward the enzyme surface. In one aspect of the invention described herein, DNA polymerases comprising phosphate region mutations exhibit improved utilization of phosphate-labeled dNTPs. Identification of the phosphate region of a particular polymerase is based on protein structures, which may be obtained from published databases of known structures, by x-ray crystallography, or by structural alignment to known structures.

A description of the method used to identify the phosphate region of DNA polymerases is provided in Example 3. The amino acid residues which comprise the phosphate regions of several specific DNA polymerases are listed in Table 5, below. The single-letter amino acid code and residue numbers are used to identify the regions. Letters next to the various listed regions indicate whether the secondary structure in that region is a part of a random coil (c), an α-helix (h), or β strand (s).

TABLE 5

| 9N (RB69DNA)* | | Taq | | Pol Beta | | Pol Eta | | HIV-RT | |
|---|---|---|---|---|---|---|---|---|---|
| (1QHT.PDB) | | (1QTM.PDB) | | (2BPF.PDB) | | (1JIH.PDB) | | (1RTD.PDB) | |
| c | Y261-V263 | s | G603-S612 | c | E147-R149 | s | 25-M31 | c | K1-V10 |
| h | I264-T267 | c | Q613 | s | I150-R152 | c | N32-A33 | c | P14-T27 |
| c | I268-T274 | h | I614-L622 | h | E153-K168 | h | F34-C43 | h | E28-E44 |
| c | E325-F327 | c | S623-D625 | c | G179-A185 | s | S58-S63 | c | G45 |
| h | P328-I337 | h | E626-E634 | s | E186-S188 | h | Y64-K68 | s | K46-S48 |
| h | L341-V344 | c | G635-D637 | c | T273-S275 | c | Y69-T76 | c | K49-T58 |
| c | S345-S347 | h | I638-F647 | h | D276-K289 | h | I77-K83 | s | P59-K65 |
| h | S348-K363 | c | G648-D655 | c | E309-E316 | c | C84-L87 | c | K66-T69 |
| h | S407-T415 | h | P656-Y671 | h | Q317-I232 | h | K268-N276 | s | K70-D76 |
| h | F448-K468 | h | A675-L682 | c | Q324-E335 | c | Y277-D281 | c | G112-S117 |
| h | P473-L489 | h | Y686-Q698 | | | s | A282-V286 | s | V118-K126 |
| c | A490-G498 | c | S699-P701 | | | c | C297-E310 | c | Y127-A129 |
| h | A553-N568 | h | K702-R717 | | | | | s | F130-I132 |
| c | P569-L577 | h | A796-G809 | | | | | c | P133-G141 |
| s | E578-V589 | c | V810-L817 | | | | | s | K142-L149 |
| | | s | E818-W827 | | | | | h | I195-R211 |
| | | | | | | | | c | W212-P226 |

*Amino acid numbering references the indicated protein database files.

### III. Native DNA Polymerases

Thermophilic DNA polymerases, like other DNA polymerases, catalyze template-directed synthesis of DNA from nucleotide triphosphates (NTPs). A primer having a free 3′ hydroxyl is required to initiate the synthesis of the DNA strand. The DNA polymerases also require divalent metal ions to function. Native thermophilic DNA polymerases have maximal catalytic activity at about 70° C. to about 80° C. At lower temperatures their activity is reduced. For example, at 37° C., many DNA polymerases have only about 10% of their maximal activity. DNA polymerases lacking 3′→5′ proofreading exonuclease activity have higher error rates than the polymerases with exonuclease activity. Table 6 depicts the nucleic acid and amino acid sequences of several exemplary native and/or wild-type DNA polymerases.

Taq DNA polymerase is a highly thermostable DNA polymerase of the thermophilic bacterium Thermus aquaticus. Taq DNA Polymerase catalyzes 5′=>3′ synthesis of DNA. The enzyme has no detectable 3′=>5′ proofreading exonuclease activity, and possesses low 5′=>3′ exonuclease activity. Native Taq DNA Polymerase is preferred for amplifications of bacterial DNA sequences homologous to those found in E. coli. The error rate of Taq polymerase is between about 1×10⁻⁴ to 2×10⁻⁵ errors per incorporated base.

Pfu DNA polymerases is derived from the bacterium Pyrococcus furiosus and has the lowest error rate of thermophilic DNA polymerases. Its error rate is about 1.5×10⁻⁶ per base pair. Besides that, Pfu DNA polymerase is highly thermostable and possesses 3′ to 5′ exonuclease proofreading activity that enables the polymerase to correct nucleotide-misincorporation errors.

The native 9°N DNA polymerase is purified from a strain of E. coli that carries a modified 9°N DNA Polymerase gene (see Southworth et al. (1996) Proc. Natl. Acad. Sci. USA 93:5281-5285) from the extremely thermophilic marine archaea Thermococcus species, strain 9°N-7. The archaea is isolated from a submarine thermal vent, at a depth of 2,500 meters, 9° north of the equator at the East Pacific Rise. The native 9°N DNA polymerase has 3′→5′ proofreading exonuclease activity. A 9°N DNA polymerase sequence is provided in Table 6.

The native Tli DNA polymerase (see Table 6) is derived from the hyperthermophile archaea Thermococcus litoralis. This polymerase (also referred to as Vent® DNA polymerase) is extremely thermostable and contains a 3′→5′ exonuclease activity that enhances the fidelity of replication. The extension rate of this enzyme is in the order of 1000 nucleotides per min. In addition, the synthesis by the polymerase is largely distributive, which can generate products of at least 10,000 bases. A two-amino acid substitution within the conserved exonuclease domain abolishes both double and single strand-dependent exonuclease activity, without altering kinetic parameters for polymerization on a primed single-stranded template (see Kong et al. (1993) J Biol. Chem. 25; 268(3): 1965-75).

Another extremely thermostable native DNA polymerase (also known as DeepVent® DNA polymerase) (see Table 6) is purified from a strain of E. coli that carries the Deep Vent DNA polymerase gene from Pyrococcus species GB-D (see Xu et al. (1993) Cell 75:1371-1377). The native organism is isolated from a submarine thermal vent at 2010 meters (see Jannasch et al. (1992) Appl. Environ. Microbiol. 58:3472-3481) and is able to grow at temperatures as high as 104° C.

The native KOD1 DNA polymerase (see Table 6) is derived from the archaeon Thermococcus kodakaraensis, strain KOD1. This DNA polymerase contains a 3′→5′ exonuclease activity and two in-frame intervening sequences of 1,080 bp (360 amino acids; KOD polymerase intein-1) and 1,611 bp (537 amino acids; KOD polymerase intein-2), which are located in the middle of regions conserved among eukaryotic and archaeal alpha-like DNA polymerases. The KOD1 DNA polymerase exhibits an extension rate (100 to 130 nucleotides per second) which is 5 times higher than that of Pfu DNA polymerase. Further, KOD1's processivity (persistence of sequential nucleotide polymerization) is 10 to 15 times higher than that of Pfu DNA polymerase (see Takagi et al. (1997) Appl. Environ. Microbiol. 63(11): 4504-4510).

Those of skill in the art will recognize that other DNA polymerases will represent polymerase sequences which are suitable for modification according to the methods and principles described herein. For example, E. coli Klenow polymerase and phi29 DNA polymerase (see Table 6) are two non-thermostable Family B polymerases which may be mutated to alter their kinetics of nucleotide incorporation.

### IV. The Mutant DNA Polymerases

The instant invention provides novel and active mutant DNA polymerases that possess altered kinetics for incorporating phosphate-labeled nucleotides during polymerization. Some of the mutants substantially lack exonuclease activity. As such, the mutant polymerases exhibit a faster or slower incorporation kinetics for deoxynucleotide-triphosphates (dNTPs) or phosphate-labeled deoxynucleotide-triphosphate (dNTP) during polymerization of DNA strands in comparison to native DNA polymerases, depending on the method used. In a preferred embodiment, the mutant polymerases are used for single-molecule sequencing or genotyping and exhibit incorporation kinetics that differ from the kinetics of polymerases which lack the mutations. In another preferred embodiment, the mutant DNA polymerases of the instant invention contain one or more mutation(s) (e.g., point mutations) in their polypeptide sequence. The mutant DNA polymerases are derived from wild-type polymerases or so-called native polymerases, such as those described herein.

Table 7 lists, in alternating fashion, the nucleic acid and amino acid sequences of more than 300 mutant DNA polymerases (SEQ ID NOs: 1-750) derived from a 9°N DNA polymerase, referred to herein as 9°N-A485L polymerase (SEQ ID NO: 2). 9°N-A485L polymerase is identical to the 9°N-Native polymerase (SEQ ID NO: 786) except that the native polymerase includes an alanine (“A”) residue at amino acid position 485, where the polymerase of SEQ ID NO:2 includes a leucine. The odd-numbered SEQ IDs in Table 7 are nucleic acid sequences and each nucleic acid sequence is followed immediately by the amino acid sequence of the mutant DNA polymerase it encodes. For example, the 9°N-A485L amino acid sequence encoded by SEQ ID NO: 1 is described by SEQ ID NO: 2, and includes a mutation at amino acid position 485, wherein the alanine (A) at position 485 in the native 9°N sequence has been changed to leucine (L). SEQ ID NO: 4 comprises the A=>L mutation at position 485, as well as an additional mutation at position 336, where the leucine (L) at position 336 in 9N-A485L has been changed to an arginine (R).

Some of the mutant DNA polymerase sequences provided herein comprise histidine tags for facilitating their purification. Where SEQ ID NOs. of polymerases comprising a histidine tag are referred to, the polymerase sequences are intended to include the sequence of the isolated polymerase without the histidine tags, as well as polymerases with the histidine tags attached.

Mutant polymerases can also contain inserted or deleted sequences when compared to the sequences of the polymerase(s) in the organisms from which they are derived. For example, SEQ ID NO: 56 includes the inserted sequence, REAQLSEFFPT, at position 329 of 9°N-A485L, and another insert, PIKILANSYRQRW, at position 485 of 9°N-A485L. Mutant DNA polymerases lacking exonuclease activity, such as the mutant 9N polymerases in Table 1, may contain 2 additional mutations at positions 141 and 143, wherein aspartic acid (D) and glutamic acid (E) are replaced with alanine (A).

Table 1 present a summary of the positions and identity of amino acid point mutations and inserts in over several hundred different mutant 9N DNA polymerases. The location of mutated amino acid residues in the polymerases of Table 1 are indicated by reference to the sequence of 9N-A485L, which differs from the 9°N-Native sequence at one position (A485). A change in amino acid sequence relative to 9°N-A485L is indicated in Table 1 by the appearance in a column cell of a letter corresponding to the amino acid which appears at the indicated position (residue positions relative to 9°N-A485L are indicated in the top row of each column). A dash (“−”) in a cell in Table 1 indicates that the identity of the amino acid at the position indicated in the column header is unchanged relative to the identity of the amino acid at the same or homologous position in 9N-A485L. Similarly, the amino acid positions of mutant polymerases 4-750 in Table 1 which are not set forth in the column headings are identical to those in the same or homologous positions in 9N-A485L.

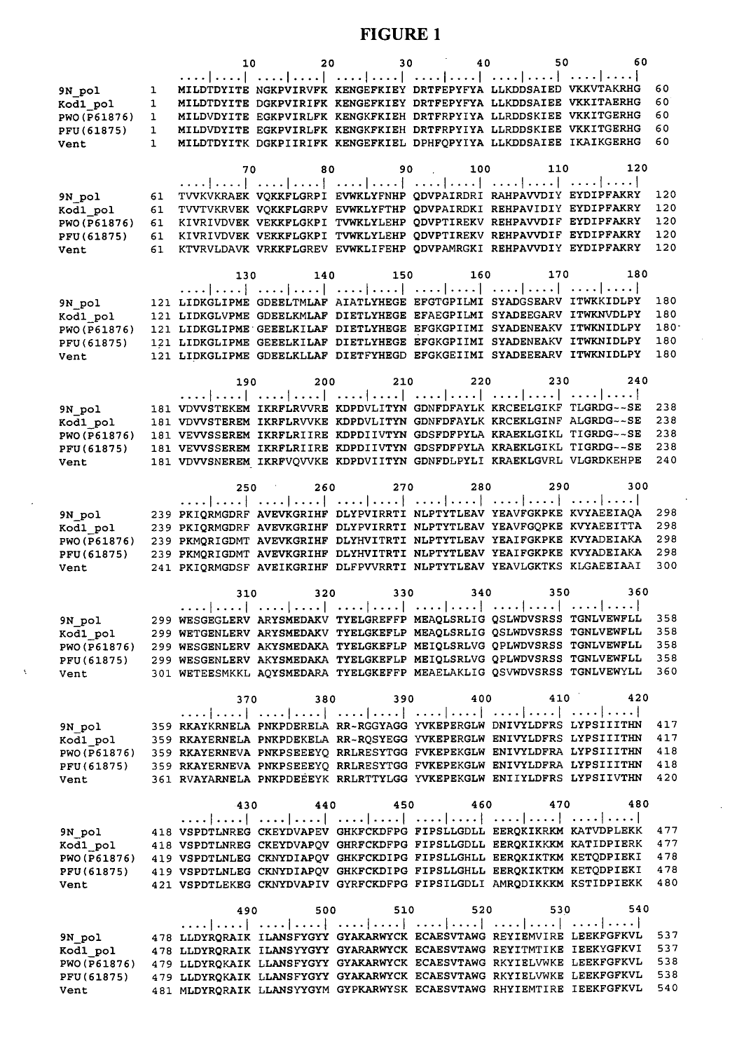

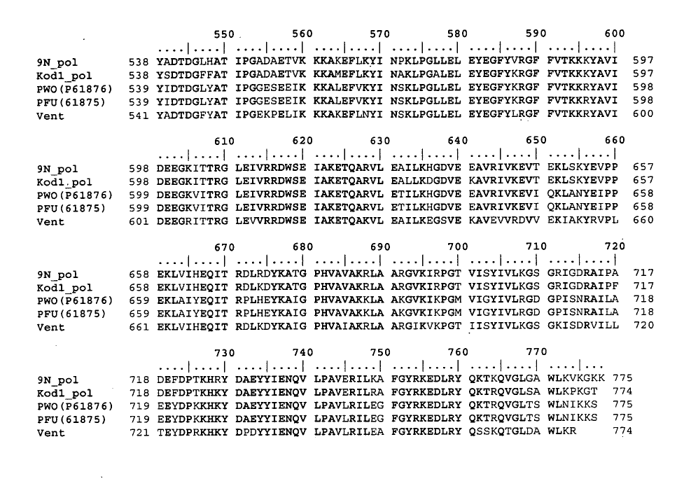

DNA polymerases can be classified into families based on segmental amino acid sequence similarities (Ito et al. (1991) Nucleic Acids Research 19:4045-4057). Homologous regions can be identified within and between polymerase families using both sequence and structural alignments (Joyce et al. (1995) Journal of Bacteriology 177(22):6321-6329). Such alignments permit the identification of corresponding amino acid positions between polymerases, both within the same polymerase family and between families. A comparison and alignment of three-dimensional structures allows for the identification of structurally homologous regions across polymerase families, and can be used apart or in conjunction with sequence alignments. As an example, FIG. 1 shows an alignment of the amino acid sequence of various family B DNA polymerases suitable for modification according to the methods and principles described herein.

The mutant DNA polymerases can be derived from various native parent enzymes, including, but are not limited to, native 9°N DNA polymerase derived from Thermococcus species 9°N-7; native Tli DNA polymerase derived from Thermococcus litoralis; native DNA polymerase derived from Pyrococcus species GB-D; native KOD1 DNA polymerase derived from Thermococcus kodakaraensis; native Taq DNA polymerase derived from Thermus aquaticus; native Phi-29 polymerase derived from Bacillus subtilis phage phi-29; and polymerase I Klenow fragment derived from the bacterium Escherichia coli.

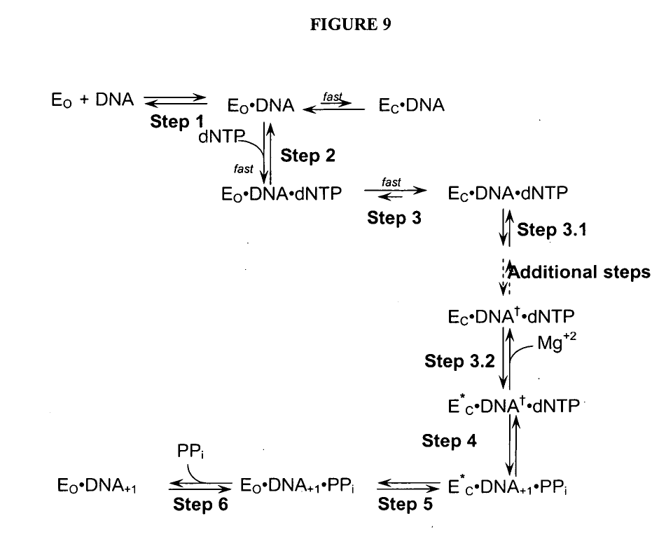

During DNA synthesis, DNA polymerase binds to a template primer and the appropriate dNTP binds with the polymerase-DNA complex. A nucleophilic attack results in phosphodiester bond formation and release of pyrophosphate (PPi). Generally, DNA binding and nucleotide binding occur rapidly. The rate-limiting step is either phosphodiester bond formation or a conformational change that precedes nucleotide incorporation. In order for nucleotides to be incorporated, it requires a dynamic interaction between the polymerase with its nucleic acid and dNTP substrates (FIG. 9). Polymerases undergo conformational changes during the DNA binding step; after the dNTP binding step and prior to chemical catalysis; after nucleotide incorporation during PPi release; and during translocation towards the new primer 3′-OH terminus (see, e.g., Patel et al. (2001) J. Mol. Biol. 308:823-837).

Polymerization involves the association of the DNA polymerase with the template primer. According to polymerase crystal structure comparisons, the thumb subdomain of the polymerase wraps around the DNA. More specifically, the thumb subdomain rotates towards the palm subdomain, and the conserved amino acid residues located within the tip of the thumb domain rotate in the opposite direction relative to the rest of the thumb such that the tip is in proximity to the DNA. These changes result in an approximately 30 angstrom (Å) wide cylinder that almost completely engulfs the DNA and the conserved amino acid residues within the tip of the thumb subdomain grip the DNA along the minor groove. The polymerase interacts primarily with the sugar-phosphate DNA backbone along the minor groove. These interactions are associated with bending of the DNA such that it adopts an S-shaped conformation. Another conformation change occurs during dNTP binding, wherein three steps are important to achieve the “induced-fit” model for nucleotide incorporation. In the first step, structural elements within the finger domain rotate toward the 3′ primer terminus, resulting in a “closed” structure. In the second step, the template base rotates back into the helix axis by greater than or equal to 90°. In the third step, the base portion of the incoming nucleotide forms a Watson-Crick base-pair with the template base, and the triphosphate portion forms metal-mediated ionic interactions with amino acid residues of the active site. The induced-fit model for nucleotide incorporation can explain how the following three interactions with the incoming nucleotides are formed during dNTP binding, namely, there is hydrogen bonding with the template base; there are stacking interactions with planar ringed amino acid residues; and there are electrostatic interactions with negatively charged phosphate groups and charged side-chains. Thus, the induced fit model appears to allow establishment of stacking interactions and also appears to serve to bring the dNTP α-phosphate close to the primer 3′-OH group, thereby promoting metal-catalyzed transfer of a nucleotide monophosphate from the dNTP to the 3′-end of the primer strand. This induced-fit mechanism for nucleotide selection also appears to restrict conformations and structures of the incoming nucleotides, promoting efficient and correct nucleotide incorporation (Patel et al., supra).

### V. A Mutant DNA Polymerase Assay

An assay system has been established for identifying the mutant DNA polymerases of the instant invention. For example, a candidate mutant DNA polymerase can be tested in a primer extension assay to determine the nucleotide incorporation rate of the mutant polymerase. Briefly, this system utilizes an oligonucleotide template, a 5′-fluorescent dye labeled oligonucleotide primer, and γ-phosphate PEG-labeled dNTPs. A mutant DNA polymerase is added to the reaction mixture and the sample is incubated at 74° C. for a fixed time (e.g., 30 sec). The reaction is stopped by adding EDTA and the average number of bases added to the primer is determined by quantifying bands on a fluorescence-based electrophoresis instrument (e.g., LI-COR 4200). This analysis provides the average nucleotide incorporation rate (nt/sec). Kinetic constants are determined by measuring incorporation rate as a function of nucleotide concentration as previously described (Kong, H. et al., J. Biol. Chem., 268(3): 1965-75 (1993)). An alternative primer extension assay, especially useful for high-throughput screening, is also disclosed herein. Mutant DNA polymerases that extend the primer faster than the native parent polymerases are selected.

Another system useful for testing DNA polymerase properties and kinetics is disclosed in U.S. Pat. No. 5,352,778, which describes an assay wherein polymerase activity is measured by the incorporation of radioactively labeled deoxynucleotides into DNAse-treated-, or activated DNA. Following subsequent separation of the unincorporated deoxynucleotides from the DNA substrate, polymerase activity is proportional to the amount of radioactivity in the acid-insoluble fraction comprising the DNA (for a detailed description see also Lehman et al. (1958) J. Biol. Chem., 233:163).

### VI. Overview of Electrokinetic Sequencing

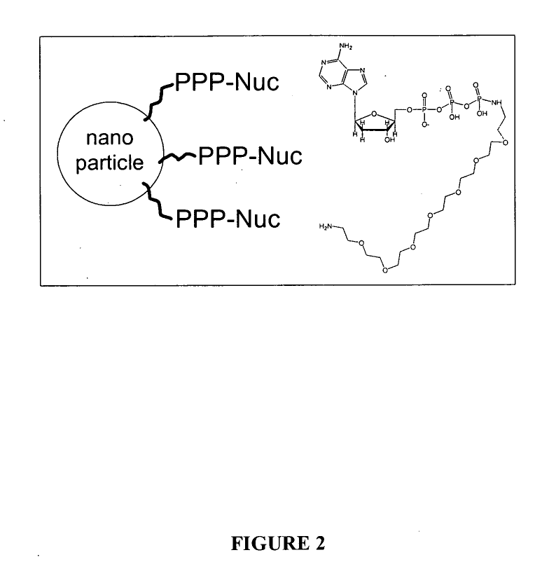

Nanoparticle Nucleotides. The mutant DNA polymerases of the instant invention can be used in electrokinetic sequencing which, in one embodiment, is based on a nucleotide configuration in which nucleotide triphosphates (NTPs) such as deoxyribonucleotide triphosphates (dNTPs) are attached to nanoparticles by a linker (FIG. 2). In one embodiment, the γ-phosphate group of the NTP can be tethered via a free-jointed linker to the surface of the nanoparticle. In a preferred embodiment, the free-jointed linker is a polyethylene glycol (PEG) linker. In certain instances, up to about 100 NTPs (e.g., dNTPs) cover the surface of a nanoparticle (e.g., 55 nm particle). Exceptionally bright fluorescence from these nanoparticles enables a charged-couple device (CCD) camera to image from about 200-300 single DNA molecules simultaneously with millisecond exposure times. In addition to improved detectability, the nanoparticles are also capable of carrying a substantial electric charge. Both characteristics, i.e., strong fluorescence and electric charge, are elements of electrokinetic sequencing methods.

Electrokinetic Cycle. In certain aspects, electrokinetic sequencing methods of the present invention comprises cycled transport of nanoparticle nucleotides between a bottom electrode and a top electrode (FIG. 2). In one embodiment, the bottom electrode is the glass bottom of a microtiter well coated with electrically-conductive, optically-transparent indium-tin oxide (ITO). About 200-300 single, individual, optically resolved polymerase-DNA complexes are immobilized in the field of view at random positions on the bottom of the well, such that the majority of complexes are optically resolvable from their nearest neighbors. This allows about 200-300 different molecules to be sequenced simultaneously by imaging a 100 μm field with a CCD camera.

In certain preferred aspects, the sequencing cycle comprises a wave of particles, which is cycled between electrodes by an alternating electric field (E-field). First, particles are concentrated in a monolayer at the bottom electrode to blanket the immobilized polymerase-DNA complexes. This allows polymerases to bind the correct nucleotides for incorporation into DNA. Next, the E-field is reversed to transport unbound particles away from the surface, leaving only particles retained by the polymerases. With unbound particles now cleared from the surface (e.g., an 800 nm distance is sufficient), retained particles are imaged by evanescent wave excitation with millisecond time resolution while the catalytic reaction is in progress. Images are acquired before the catalytic reaction is completed because, after incorporation of the nucleotide into DNA, pyrophosphate and the attached nanoparticle are released from the enzyme. This completes one sequencing cycle. The timing of E-field switching and image acquisition is dictated by the duration of the catalytic cycle, and is expected to range from about 1-100 msec (Levene et al., Science, 299:682 (2003)).

Throughput. In one embodiment, when the electrokinetic cycle operates at 10 cycles/sec (i.e., 100 msec period), the maximum possible sequencing speed (or catalytic rate) is 10 bases per second. At this speed, a 20 kb DNA molecule can be sequenced in 33 min with any mutant DNA polymerase of the instant invention. In addition, net throughput can be significantly enhanced by multiplexing. For example, with an average of one polymerase-DNA complex per 50 μm² area, there are about 200 optically-resolved complexes in the optical field (100×100 μm) in the bottom of the microtiter well. In this embodiment, each well is used only once for a period of 33 min. to simultaneously sequence all 200 20 kb DNA molecules (i.e., 4 million bases total). Then, the next 4 million bases can be sequenced in a new well, and so on. Although it would take about 30-40 days to process one 1536 well plate, one well at a time, the plate can be processed 4 times faster (i.e., in 7-10 days) by quadruplexing the instrument optics. Under these conditions, a 1536 well microtiter plate can produce the equivalent of 2 human genomes worth of sequence (i.e., 4 million bases/well×1536 wells=6.1 billion bases total).

### VII. Methods for Long-Read Single Molecule Sequencing

Methods for long-read sequencing that employ the mutant DNA polymerases of the instant invention generally fall into two categories, depending on whether fluorescence or electrical detection is used. Fluorescence methods monitor either nucleotide addition by polymerase or exonucleolytic hydrolysis of prelabeled DNA. Polymerase long-read methods use phosphate-labeled nucleotides that are released after base incorporation.

Electrokinetic sequencing is an example of a single molecule sequencing method. This method utilizes dNTPs modified with a dye label on the phosphate. The labeled phosphate released after base addition allows the label to be detected before, during or after separation from unused nucleotides in a microfluidics system.

The use of 50 nm zero-mode waveguides (i.e., 50 nm diameter apertures in a metal film) for near-field detection of phosphate-labeled nucleotides bound to mutant polymerases of the instant invention during the catalytic cycle is another example of a single molecule sequencing method (see also Levene et al., Science, 299:682 (2003)). The waveguide allows the enzyme to be detected in a small volume without interference from labeled nucleotides in bulk solution. High-throughput sequencing (i.e., imaging of 200-300 polymerases simultaneously with a CCD camera) is advantageously provided by the electrokinetic sequencing methods described herein.

A third method for single molecule sequencing involves labeling the mutant DNA polymerase with a fluorophore and detecting modulation of the fluorescence signal by fluorescence resonance energy transfer (FRET) as phosphate-labeled nucleotides, labeled with quenchers or other fluorophores, transiently bind to the enzyme. Background signal from nucleotides in the bulk medium is reduced by detecting modulation of the enzyme fluorescence, instead of directly detecting the nucleotide label. Herein, the polymerase is exposed to continuous illumination.

Non-fluorescent sequencing methods propose to detect electric signals from individual bases as a DNA strand traverses through a nanopore (Deamer et al., Acc. Chem. Res., 35:817 (2002)) and can be employed with the mutant DNA polymerases of the instant invention.

The electrokinetic sequencing method that employs the mutant DNA polymerases of the present invention overcomes the limitations and challenges of other single molecule sequencing methods. In addition, the mutant DNA polymerases, such as those described in Tables 1-3 which exhibit increased rates of phosphate labeled nucleotide incorporation, are suitable for use in the immobilized polymerase-DNA complexes described herein. As such, electrokinetic sequencing provides long-read high-throughput sequencing with sufficient resolution and without the need to label the polymerase.

### VIII. Topologically Linked Polymerase-DNA Complexes

In a preferred embodiment, the polymerase-DNA complexes are taught and described in U.S. Patent Publication No. 2005/0042633, published Feb. 24, 2005, and incorporated herein by reference. As described therein, a polymerase-nucleic acid complex (PNAC), comprises: a target nucleic acid and a nucleic acid polymerase, wherein the polymerase has an attachment complex comprising at least one anchor, which at least one anchor irreversibly associates the target nucleic acid with the polymerase to increase the processivity index. As used herein, the term “processivity index” means the number of nucleotides incorporated before the polymerase dissociates from the DNA. Processivity refers to the ability of the enzyme to catalyze many different reactions without releasing its substrate. That is, the number of phosphodiester bonds formed is greatly increased as the substrate is associated with polymerase via an anchor.

In a preferred embodiment, the polymerase is attached to the ITO permeation layer and stably associated with a DNA template to achieve long sequence reads. The polymerase can be attached to the ITO permeation layer via various linkages including, but not limited to, covalent, ionic, hydrogen bonding, Van der Waals' forces, and mechanical bonding. Preferably, the linkage is a strong non-covalent interaction (e.g. avidin-biotin) or is covalent. In order to permanently associate the DNA template and the polymerase to the ITO permeation layer, an approach that functionally mimics the sliding clamp of a replisome, as described in Shamoo et al., Cell, 99:155 (1999), can be used.

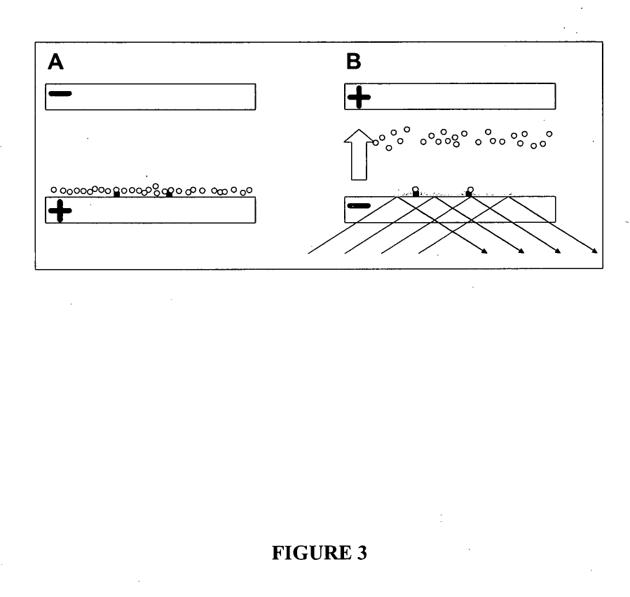

As shown in FIG. 3, the polymerase-DNA complex is covalently attached (i.e., anchored) to the ITO permeation layer through two linkers in order to irreversibly capture the DNA while still allowing it to slide through the polymerase active site. Circular in form, the DNA (˜20 kb) is topologically linked to the immobilized polymerase, and therefore does not dissociate.

The methods of the present invention employ a mutant DNA polymerase such as a mutant DNA polymerase I, II, or III. Preferably, the methods employ a mutant DNA polymerase derived from family B polymerases. Suitable family B polymerases include, but are not limited to, a 9°N DNA polymerase derived from Thermococcus species 9°N-7; a Tli DNA polymerase derived from Thermococcus litoralis; a DNA polymerase derived from Pyrococcus species GB-D; a KOD1 DNA polymerase derived from Thermococcus kodakaraensis; a Taq DNA polymerase derived from Thermus aquaticus; a Phi-29 polymerase derived from Bacillus subtilis phage phi-29; and a polymerase I Klenow fragment derived from the bacterium Escherichia coli. Specific examples include, but are not limited to, any of the mutant DNA polymerases set forth in SEQ ID NO: 4 through SEQ ID NO: 750 (9°N mutants), SEQ ID 754-764 (Klenow mutants) or SEQ ID NO: 767 through SEQ ID NO: 784. Those of skill in the art will know of other enzymes or polymerases suitable for use in the present invention.

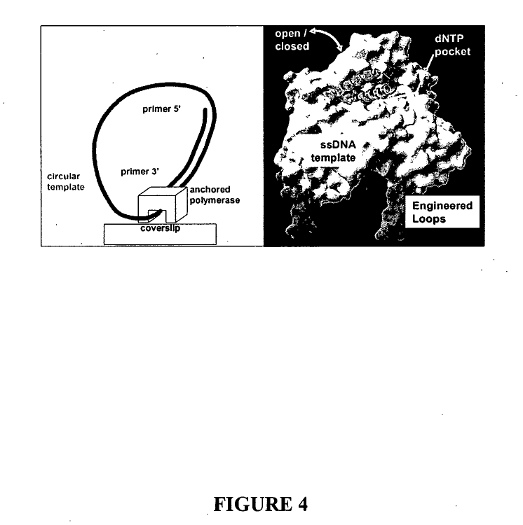

Examples of modified DNA polymerases that can be used with the methods of the instant invention are mutants derived from 9N-A485L (SEQ ID NO: 2; commercially available as Therminator™ (New England Biolabs, Inc)), including those listed in Table 1. The protein regions on either side of the DNA binding cleft of 9N-A485L, likely to be conformationally rigid, were identified based upon previous studies with RB69 polymerase. Loops of ten amino acids containing a 6×His sequence at five candidate positions were inserted. Loops inserted at positions K53 and K229 of 9N-A485L (modeled in FIG. 4) had no measurable effect on polymerase activity when present either individually or combined, and both were capable of binding Ni-NTA beads in an affinity purification procedure. Based upon previous studies on active immobilized polymerases and other enzymes, e.g., unoriented T7 DNA polymerase (Levene et al., Science, 299:682 (2003)), oriented EcoRI (Bircakova et al., J. Mol. Recognit., 9:683 (1996)), and solid-phase enzymes used in bioprocess engineering (Berg et al., In “Interfacial Enzyme Kinetics,” John Wiley & Sons (2002)), the engineered polymerase is expected to display activity on a surface. Alternatively, non-covalent bonding is employed for attaching the polymerase via the 6×His loops to a Ni-NTA-activated permeation layer.

Suitable covalent coupling methods include, without limitation, a maleimide or thiol-activated permeation layer coupled to specific cysteine amino acids on the polymerase surface, a carboxylate permeation layer coupled to specific lysine amino acids on the polymerase surface, a hydrazine permeation layer coupled to the unnatural amino acid p-acetyl-L-phenylalanine on the polymerase surface, and the like. The latter is particularly interesting because of its high coupling specificity, long reactant shelf life, and imminent commercialization (Wang et al., PNAS, 100:56 (2003)). Given a suitable coupling chemistry, complexes can be formed by mixing the 9N-A485L polymerase with primed circular DNA and driving them electrically to the electrode surface for covalent coupling. To ensure that most anchored 9N-A485L proteins are associated with template, DNA is used at concentrations exceeding the binding constant (K_{mDNA}=50 pM for 9N; New England Biolabs Catalog). Polymerases anchored without DNA are neglected because they have no sequencing activity. When polymerase attachment is complete, the electric field is reversed to elute linear (e.g., broken) DNA templates, such that the only anchored polymerases capable of generating sequence data are those complexed with circular DNA templates. A simple computer model indicates that 200-300 polymerase-DNA complexes can be dispersed randomly in a 100 μm field of view at optically resolvable distances. The number of resolvable complexes decreases at higher densities because of overcrowding. Random dispersion on an inexpensive ITO surface provides an easy way to isolate single molecules for multiplexed, long-read sequence analysis. In another embodiment, polymerases are allowed to bind non-specifically to the ITO surface, rather than binding by specific anchors. Some of the polymerases will bind in an inactive orientation, and others will bind in an active orientation. Particularly, only those bound in an active orientation will produce signals from the sequencing reaction, while those bound in an inactive orientation will not produce signals and will therefore be undetectable.

### IX. Examples

The following examples are offered to illustrate, but not to limit, the claimed invention.

### Example 1

### Library Screening Method

This example illustrates the screening of a mutant DNA polymerase library. The cDNA library was constructed by cloning genes of DNA polymerases (i.e., 9N-A485L DNA polymerase (SEQ ID NO: 2) and 9°N-Native DNA polymerase) into expression plasmids. The polymerase genes were mutated at specific nucleotide positions to create the mutant DNA polymerases (see Table 1 and the sequences of Table 7). A primer extension assay was used to estimate the polymerase activity of the various mutants that were generated.

Library Construction. Therminator™ DNA polymerase (i.e., 9N-A485L; SEQ ID NO: 2) and 9°N-Native DNA polymerase genes were obtained from New England Biolabs. The genes were cloned into an arabinose-inducible expression plasmid (pBAD, Invitrogen). Mutations were introduced at specific nucleotide positions using the QuikChange™ site-directed mutagenesis kit according to the manufacturer's instructions (Stratagene). Preferably, all three nucleotides of a target codon were randomized using a single degenerate oligonucleotide in order to generate the mutant DNA polymerase library containing all 20 amino acids at that position. Multiple codons were randomized using multiple degenerate oligonucleotides targeting multiple sites in a single mutagenesis reaction (Stratagene), or by randomizing a second position starting from a library already randomized at a first position.

Protein Expression And Extraction. Single colonies of library clones were grown overnight in 96-well plates containing 100 μl of growth medium per well. The clones were subcultured by diluting (overnight) cultures 100-fold into a fresh culture plate containing 100 μl of growth medium supplemented with 0.04% arabinose to induce protein expression. After 4 hour growth at 37° C., 20 μl of lysis solution (10 mM Tris Cl pH 8, 0.8% IGEPAL) was added to each well, the plate was sealed with foil and heated at 75° C. for 10 min. Cell lysates were stored at 4° C. for up to 2 weeks with little loss of thermophilic polymerase activity.

Polymerase Assay. A primer extension assay was used to estimate polymerase activity. The primer is 5′-labeled with a fluorophore (e.g., FAM), and the template is 3′-labeled with a quencher (e.g., Black Hole Quencher I, Biosearch Technologies). Primer extension by polymerase was estimated by melt-curve analysis of the template-primer duplex using a real-time PCR instrument (Opticon I, MJ Research). The fluorescence signal increased as the duplex melted and the fluorescent primer strand separated from the template strand. As the primer is extended by polymerase, the Tₘ increases. Reactions were performed in duplicate, with one sample (96-well plate) containing unlabeled nucleotides and the other PEG-labeled nucleotides. Each mutant was scored by taking the ratio of activity between unlabeled and PEG-labeled nucleotides, in order to normalize for variation in the amount of polymerase in each well. Mutants showing a higher ratio of activity with PEG-labeled in comparison to unlabeled nucleotides were selected for further characterization. Alternatively, if the protein amounts were sufficiently uniform from sample to sample, improved mutants were selected based on their activity with PEG-labeled nucleotides alone.

Reactions contained 0.1-5.0 μl cell lysate, 0.2% NP-40 (contributed by cell lysate and supplemented as necessary, depending on lysate volume), 20 mM Tris-Cl pH 9.2, 50 mM KCl, 5 mM MgSO₄, 150 nM template (5′-CGGCTGCCTGGCGCGTCGGAGTGCTCA), 100 nM primer (5′-FAM-TGAGCACTCCGACGCGCCA), and either unlabeled nucleotides or PEG-labeled nucleotides. A chemically synthesized “full length” primer (5′-FAM-TGAGCACTCCGACGCGCCAGGCAGCCG) is utilized in a control sample as explained below. Preferably, unlabeled nucleotides were used at 1 μM each, and PEG-labeled nucleotides were at 200 μM each; a mixture of all four nucleotides A, C, G and T was used in each reaction mix. The preferred incubation temperature was 68° C. and the preferred incubation time is 1-30 min. To capture mutant polymerases with low temperature activity a two stage incubation was performed (i.e., 40 C then 68 C). After incubation, melting data was acquired at 1° C. intervals from 65° C. to 90° C. Reaction conditions (i.e., lysate amount, nucleotide concentration, incubation temperature, incubation time) were adjusted so that the primer was partly extended, allowing detection of either increased (long extension) or decreased (short extension) activity of each tested polymerase mutant.

Analysis. Melt curves are analyzed by a software program (written in the LabVIEW G programming language, National Instruments Inc.) as follows. The raw data comprises fluorescence F at each temperature T from 65° C. to 90° C. The F values are smoothed with the standard LabVIEW median filter (window parameter=2) and the first derivative dF/dT is taken at each temperature point T along the curve. The same median filter is applied to the dF/dT values, and the resulting smoothed dF/dT values are rescaled between 0-1 such that the minimum dF/dT value is 0.0 and the maximum 1.0, with all other values in between. The rescaled (0-1) dF/dT values (at each temperature T) are summed over two user-defined temperature ranges: for example, a low temperature range 68 C to 77 C (lowRangeSum) and a high temperature range 78 C to 85 C (highRangeSum). The obtained lowRangeSum and highRangeSum values are normalized such that
lowRangeSum+highRangeSum=1.
The raw score for polymerase activity is given by
rawScore=highRangeSum−lowRangeSum
where rawScore ranges −1 to +1 (the −1 extreme if lowRangeSum=1 and highRangeSum=0; and the +1 extreme if lowRangeSum=0 and highRangeSum=1).

The rawScore data for all samples is normalized between two control samples run in the same sample set as the mutant polymerases. Both controls utilize E. coli lysates from cells that express no thermostable polymerase activity. The “unextended” control contains the same primer used in the test samples, but the primer remains unextended because there is no active polymerase. The “full extension” control contains the “full length” primer sequence defined above. Each rawScore is normalized between the controls as
activityScore=(rawScore_{—}i−noExt)/(fullExt−noExt),
where activityScore is the normalized score, rawScore_i is the rawScore for the i^{th} sample, noExt is the rawScore of the unextended control, and fullExt is the rawScore of the full extension control. The activityScore values are used to rank mutant polymerases relative to their respective parent polymerase in order to identify mutant polymerases with improved activity.

The activityScore as defined above is a highly reproducible way to rank polymerase activity. Alternative methods include, for example, determining the melting temperature from F vs T data or dF/dT vs T data (Wittwer C T, Reed G H, Gundry C N, Vandersteen J G, Pryor R J (2003) Clinical Chemistry 49: 853-860).

Results. Mutant polymerases selected from different libraries were compared on a single 96-well assay plate using 9N-A485L as a control. Scores were determined for PEG-labeled nucleotides only, without comparison to unlabeled nucleotides. The activityScore of each mutant were given relative to a control polymerease, e.g., 9N-A485L, where the activityScore of 9N-A485L has been normalized to 1.0. A relative score >1.0 indicates=d an improvement over the 9N-A485L with respect to the utilization of PEG-labeled nucleotides, while a relative score <1.0 indicated reduced activity. Corresponding rates of phosphate-labeled nucleotide incorporation were determined and the mutant 9N polymerases were sorted according to their activities relative to 9N-485L (SEQ ID NO. 2). The results are compiled in Table 1. An asterix (“*”) in the score column in Table 1 means that the relative rate of nucleotide incorporation by the mutant DNA polymerase versus 9N-485L is 0.99 or less; a “+” means that the relative rate is between 1 and 2.99; a “++” means the relative rate is between 3 and 6.99; and a “+++” means the relative rate as measured by the assay (see Example 1) is between 7 and 23 times faster.

Similar protocols were used to prepare and study phosphate region mutants of Taq DNA polymerase and Klenow DNA polymerase. Taq DNA polymerase mutants with improved rates of phosphate-labeled nucleotide incorporation are shown in Table 2, and Klenow DNA polymerase mutants with improved rates of phosphate-labeled nucleotide incorporation are shown in Table 3.

TABLE 2
Summary of Mutant Klenow DNA Polymerases

| Relative | | | | | | | | |
|---|---|---|---|---|---|---|---|---|
| Score | | 589 | 617 | 645 | 691 | 693 | 726 | nt/min |
| 1.0000 | SEQ ID NO: 766 | G | V | I | R | A | R | 1.12 |
| | Klenow | | | | | | | |
| | Polymerase Parent | | | | | | | |
| 2.2142 | SEQ ID NO: 768 | — | — | — | Y | — | — | 2.4675 |
| 2.0747 | SEQ ID NO: 770 | — | — | — | — | G | — | 2.625 |
| 2.9549 | SEQ ID NO: 772 | — | — | — | — | — | S | 1.6225 |
| 3.0248 | SEQ ID NO: 774 | D | — | H | — | — | — | 11.475 |
| 3.0393 | SEQ ID NO: 776 | — | — | F | — | — | — | 10.65 |
| 2.9283 | SEQ ID NO: 778 | — | — | H | — | — | — | 11.225 |
| 2.9905 | SEQ ID NO: 780 | — | — | K | — | — | — | 10.4 |
| 2.9660 | SEQ ID NO: 782 | — | I | — | — | — | — | 1.5275 |
| 3.1200 | SEQ ID NO: 784 | — | — | W | — | — | — | 14.725 |

TABLE 3
Summary of Mutant Taq DNA Polymerases

| Relative | | | | | | | |
|---|---|---|---|---|---|---|---|
| Score | | 395 | 423 | 469 | 471 | 504 | nt/min |
| 1.0000 | SEQ ID NO: 752 | V | I | R | A | L | 0.8414 |
| | Taq Polymerase | | | | | | |
| | Parent | | | | | | |
| 0.9705 | SEQ ID NO: 754 | C | — | — | — | — | 0.7657 |
| 1.2910 | SEQ ID NO: 756 | — | K | — | — | — | 3.6286 |
| 1.3193 | SEQ ID NO: 758 | — | E | — | — | — | 1.1457 |
| 1.1427 | SEQ ID NO: 760 | — | — | I | — | — | 0.7571 |
| 0.9403 | SEQ ID NO: 762 | — | — | — | S | — | 0.7286 |
| 1.0559 | SEQ ID NO: 764 | — | — | — | — | G | 1.2029 |

### Example 2

### Gel Extension Assay of Mutant Polymerase Activity

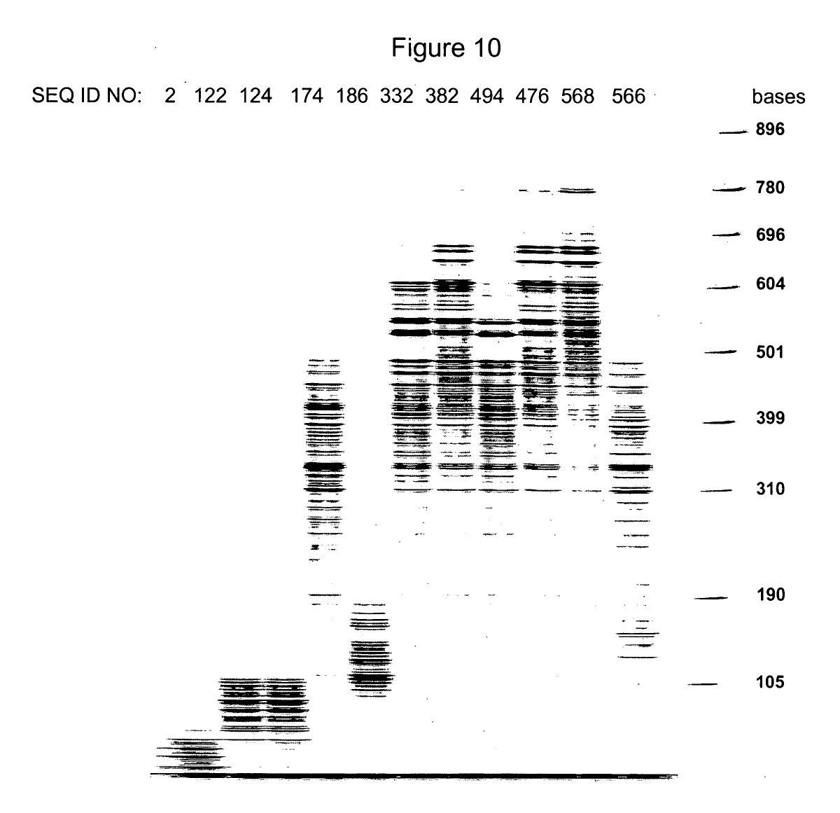

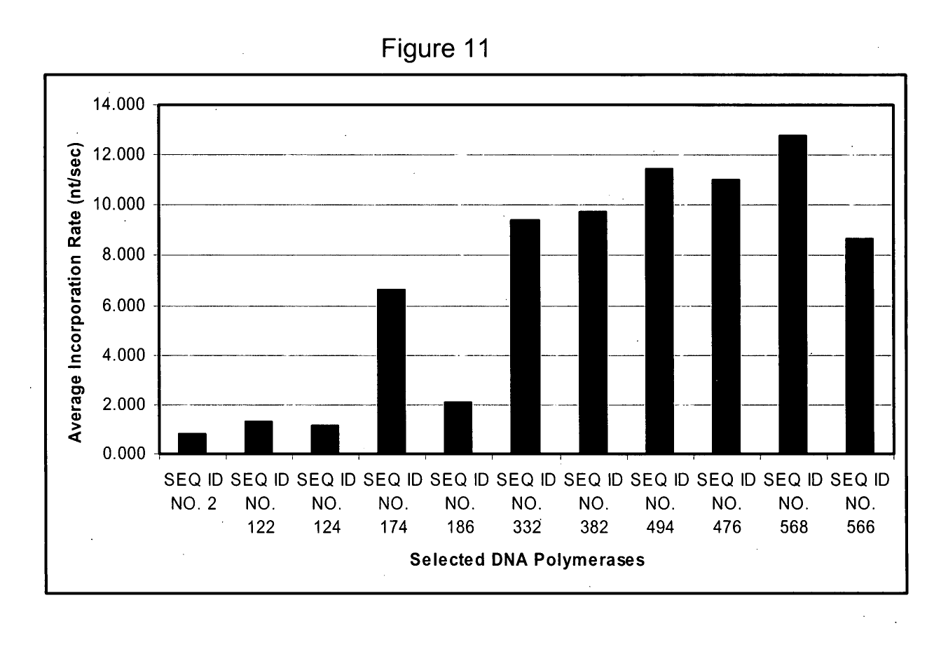

A gel extension assay using saturating amounts of selected purified mutant DNA polymerases was used to analyze their activity. Each enzyme was incubated at 68° C. for 30 seconds with an IRDye700 labeled primer hybridized to ssM13mp18 and saturating amounts of phosphate-labeled nucleotides. Reactions were resolved on a 10% TBE-Urea gel using a LI-COR 4200 DNA Analyzer. The average rate (nucleotides per second) for each of the indicated enzymes was calculated. The results are shown in FIGS. 10 and 11.

### Example 3

### Defining Phosphate Regions of DNA Polymerases

Taq DNA polymerase (Family A). Taq DNA polymerase was analyzed using public-domain software (Swiss-PDB Viewer version 3.7, http://ca.expasy.org/spdbv/). Initially, the protein (1QTM.pdb; Berman et al., Nucleic Acids Res, 28:235 (2000)) was divided into 2 regions by a plane parallel to the two paired bases in the active site (i.e., parallel to the aromatic ring moieties of both the bound dTTP and of the templating adenosine). This was accomplished in “slab” view (slab depth 100 A), by both rotating the model and translating the slab until the two bases were co-planar with the slab. The model was oriented with the phosphate groups of dTTP pointed into the display screen. The slab was then translated further into the screen to hide from view both bases as well as the alpha and beta phosphates of dTTP, so that only the gamma phosphate and amino acids between the gamma phosphate and the protein surface were visible. Then, the set of visible amino acids was further narrowed by selecting only amino acids within 15 Å of the dTTP. Secondary structure elements containing amino acids of the narrowed set define the phosphate region of Taq DNA polymerase (Table 5).

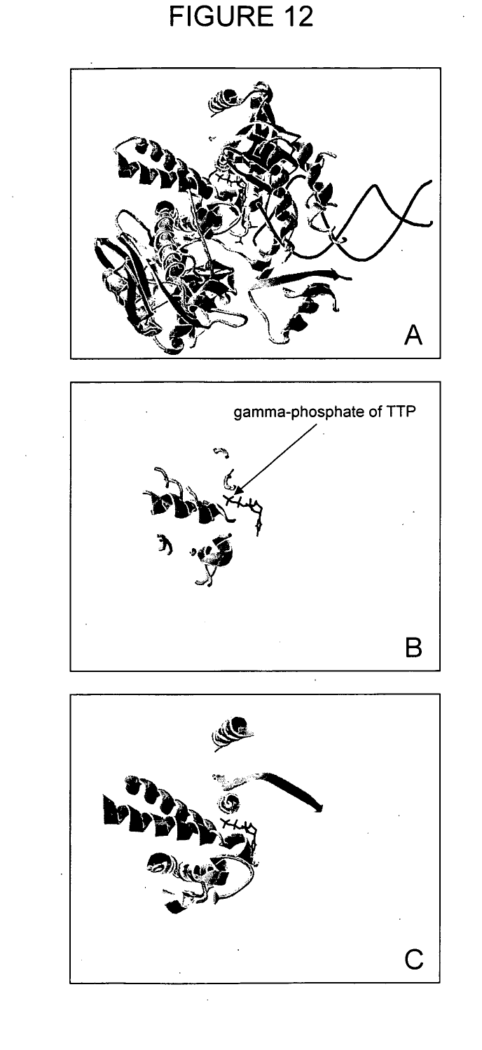

9°N polymerase (Family B). The published structure of 9°N polymerase (1QHT.pdb) does not contain bound dNTP or template DNA. These two elements were therefore modeled into 9°N holoenzyme by structural alignment with RB69 DNA polymerase (1IG9.pdb), using the structurally conserved palm domain as described for aligning polymerases eta and T7 (Trincao et al., Mol Cell, 8:417 (2001)). Four structurally conserved beta strands in the two palm domains were visually identified and aligned between RB69 (Y619-V627, G700-T703, R707-V712, K724-K726) and 9°N (Y538-A546, G586-V589, K592-I597, T604-R606) using Swiss-PDB Viewer version 3.7 (http://ca.expasy.org/spdbv/). The structures were then superimposed using the function, “Fit molecules (from selection),” giving an RMS deviation of 2.49 Å for 22 aligned alpha carbon atoms. All of the amino acids of 9°N, plus the DNA and dTTP groups from RB69, were merged into a single pdb file using the function “Create merged layer from selection” (see FIG. 12). The phosphate region was defined as explained in this Example with respect to Taq polymerase (Table 5).

Phosphate regions of polymerase beta, eta and HIV-RT (Families X, Y and RT). Published structures of polymerases beta (2BPF.pdb) and HIV-RT (1RTD.pdb) contain bound dNTP and template DNA, enabling both to be analyzed as Taq and 9°N DNA polymerase were analyzed. Their phosphate regions are given in Table 5. Polymerase eta (1JIH.pdb) was merged with template and dNTP from T7 DNA polymerase (1T7P.pdb) using the conserved palm domains provided in (Trincao et al., (2001)) for this structural alignment. The phosphate region of polymerase eta is given in Table 5.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

LENGTHY TABLE REFERENCED HERE
US20070048748A1-20070301-T00001
Please refer to the end of the specification for access instructions.

LENGTHY TABLE REFERENCED HERE
US20070048748A1-20070301-T00002
Please refer to the end of the specification for access instructions.

LENGTHY TABLE REFERENCED HERE
US20070048748A1-20070301-T00003
Please refer to the end of the specification for access instructions.

LENGTHY TABLE REFERENCED HERE
US20070048748A1-20070301-T00004
Please refer to the end of the specification for access instructions.

LENGTHY TABLE REFERENCED HERE
US20070048748A1-20070301-T00005
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070048748A1). An electronic copy of the table will also be available from the USPTO upon request and payment of the fee set forth in 37 CFR 1.19(b)(3).

What is claimed is:

1. A mutant DNA polymerase, wherein the amino acid sequence of the phosphate region of said mutant DNA polymerase comprises two or more mutations not present in the phosphate region of the most closely related native DNA polymerase, and wherein said two or more phosphate region mutations increase the rate at which said mutant DNA polymerase incorporates a phosphate-labeled nucleotide.

2. The mutant DNA polymerase of claim 1, wherein said mutant DNA polymerase, or at least the phosphate region of said mutant polymerase, is derived from a Family A or Family B polymerase.

3. The mutant DNA polymerase of claim 2, wherein said mutant polymerase is a Family B polymerase.

4. The mutant DNA polymerase of claim 3, wherein said mutant polymerase is a 9°N DNA polymerase.

5. The mutant DNA polymerase of claim 4, wherein said mutant 9°N DNA polymerase incorporates phosphate-labeled nucleotides at an increased rate relative to 9°N-A485L DNA polymerase (SEQ ID NO: 2); and
wherein said mutant 9°N DNA polymerase comprises an alanine to leucine mutation at amino acid position 485; and
wherein said mutant 9°N DNA polymerase further comprises one or more additional mutations in the phosphate region of said mutant 9°N DNA polymerase.

6. The mutant 9°N DNA polymerase of claim 5, wherein said one or more additional mutations are selected from the group consisting of a mutation at amino acid position 352, 355, 408, 460, 461, 464, 480, 483, 484, and 497, and combinations thereof.

7. The mutant 9°N DNA polymerase of claim 5, wherein said one or more additional mutations comprises a mutation at amino acid position 484.

8. The mutant 9°N DNA polymerase of claim 5, wherein said one or more additional mutations includes mutations at amino acid positions 408, 464, and 484.

9. The mutant 9°N DNA polymerase of claim 8, wherein said mutation at position 408 is selected from the group consisting of tryptophan, glutamine, histidine glutamic acid, methionine, asparagine, lysine, and alanine; and wherein said mutation at position 464 is selected from the group consisting of glutamic acid and proline; and wherein said mutation at position 485 is tryptophan.

10. The mutant 9°N DNA polymerase of claim 8, wherein said amino acids at positions 408, 464, and 484 are tryptophan, glutamic acid, and tryptophan, respectively.

11. The mutant 9°N DNA polymerase of claim 5, wherein said mutant 9°N DNA polymerase incorporates phosphate-labeled nucleotides at an increased rate relative to 9°N-A485L DNA polymerase (SEQ ID NO: 2), and wherein said rate is at least three times faster than that catalyzed by 9°N-A485L DNA polymerase.

12. The mutant 9°N DNA polymerase of claim 5, wherein said mutant 9°N DNA polymerase incorporates phosphate-labeled nucleotides at an increased rate relative to 9°N-A485L DNA polymerase (SEQ ID NO: 2), and wherein said rate is at least seven times faster than that catalyzed by 9°N-A485L DNA polymerase.

13. The mutant 9°N DNA polymerase of claim 5, wherein said mutant 9°N DNA polymerase incorporates phosphate-labeled nucleotides at an increased rate relative to 9°N-A485L DNA polymerase (SEQ ID NO: 2), and wherein said rate is at least twenty times faster than that catalyzed by 9°N-A485L DNA polymerase.

14. The mutant 9°N DNA polymerase of claim 5, wherein said mutant 9°N DNA polymerase incorporates phosphate-labeled nucleotides at an increased rate relative to 9°N-A485L DNA polymerase (SEQ ID NO: 2), and wherein said rate is at least fifty times faster than that catalyzed by 9°N-A485L DNA polymerase.

15. The mutant 9°N DNA polymerase of SEQ ID NO. 568.

16. The mutant 9°N DNA polymerase of SEQ ID NO. 568, further comprising one or more additional mutations, wherein said one or more additional mutations are selected from the group consisting of an alteration in amino acid identity, an insertion of one or more amino acids, and the deletion of one or more amino acids.

17. The mutant 9°N DNA polymerase of SEQ ID NO. 568 and conservative modifications thereof.

18. The mutant 9°N DNA polymerase of claim 17, further comprising one or more additional mutations, wherein at least one additional mutation is in the phosphate region of said mutant 9°N DNA polymerase.

19. The mutant 9°N DNA polymerase of claim 18, wherein the additionally mutated amino acid is selected from the group consisting of the asparagine at position 491 and lysine at position 487.

20. A mutant 9°N DNA polymerase with an amino acid sequence selected from the group consisting of the even-numbered SEQ ID NOs 4 through 750.

21. A purified nucleic acid sequence encoding a polymerase of claim 20.

22. A method for identifying polymerases with improved suitability for a nucleotide sequencing process, wherein the improved suitability is measured relative to that of a parent polymerase, comprising:
(1) assaying the rate of phosphate-labeled nucleotide incorporation by a test mutant polymerase, wherein said phosphate region of said test polymerase is at least 90% identical to said parent polymerase;
(2) determining if said rate of phosphate-labeled nucleotide incorporation by said test mutant polymerase is suitable for said nucleotide sequencing process; and,
if said rate of phosphate-labeled nucleotide incorporation is suitable, then identifying the test mutant polymerase as such.

23. The method of claim 22, wherein if said rate of phosphate-labeled nucleotide incorporation is not suitable, repeating steps (1) and (2) with a second test mutant polymerase until a suitable polymerase is identified.

24. The method of claim 23, wherein said second test mutant comprises each of the mutations in the previous test mutant polymerase, and further comprises at least one additional mutation relative to the previous test mutant polymerase.

25. The method of claim 22, wherein said polymerase is a thermostable polymerase.

26. The method of claim 22, wherein the amino acid sequence of said parent polymerase is at least 90% identical to the amino acid sequence of 9°N-A485L DNA polymerase (SEQ ID NO: 2).

27. The method of claim 22, wherein the amino acid sequence of said parent polymerase is at least 95% identical to the amino acid sequence of 9°N-A485L DNA polymerase (SEQ ID NO: 2).

28. The method of claim 26, wherein said improved polymerase is a polymerase which incorporates between 1 and 20 phosphate-labeled nucleotides per second.

29. The method of claim 28, wherein said improved polymerase is a polymerase which incorporates between 5 and 15 phosphate-labeled nucleotides per second.

30. The method of claim 29, wherein said nucleotide sequencing process is a field-switch polynucleotide sequencing process.

31. A mutant polymerase identified by the method of claim 24.

32. A mutant polymerase identified by the method of claim 27.

33. A mutant DNA polymerase, wherein the amino acid sequence of the phosphate region of said mutant DNA polymerase comprises one or more mutations not present in the phosphate region of the most closely related native DNA polymerase, and wherein said one or more phosphate region mutations increase the rate at which said mutant DNA polymerase incorporates a phosphate-labeled nucleotide.

34. The mutant DNA polymerase of claim 33, wherein said mutant DNA polymerase is a Family A DNA polymerase.

35. The mutant DNA polymerase of claim 35, wherein said mutant polymerase is a mutant Klenow DNA polymerase.

36. The mutant DNA polymerase of claim 35, wherein said mutant Klenow polymerase incorporates phosphate-labeled nucleotides at an increased rate relative to the Klenow DNA polymerase of SEQ ID NO: 752; and
wherein said mutant Klenow DNA polymerase comprises one or more phosphate region mutations.

37. The mutant Klenow DNA polymerase of claim 36, wherein said one or more additional mutations are selected from the group consisting of a mutation at amino acid position 423 and 504, and combinations thereof.

38. The mutant Klenow DNA polymerase of claim 37, wherein the amino acid at position 423 is mutated.

39. The mutant Klenow DNA polymerase of claim 38, wherein the amino acid at position 504 is mutated.

40. The mutant Klenow DNA polymerase of claim 39, wherein the amino acid at position 423 is lysine or glutamic acid.

41. The mutant Klenow DNA polymerase of claim 40, wherein the amino acid at position 504 is glycine.

42. The mutant Klenow DNA polymerase of claim 41, wherein said mutant polymerase incorporates phosphate-labeled nucleotides at a rate at least three times faster than the Klenow polymerase of SEQ ID NO: 752.

43. The mutant Klenow DNA polymerase of SEQ ID NO: 756, 758, or 764.

44. A purified nucleotide acid encoding a mutant Klenow DNA polymerase of claim 43.

45. The mutant DNA polymerase of claim 34, wherein said mutant polymerase is a mutant Taq DNA polymerase.

46. The mutant DNA polymerase of claim 43, wherein said mutant Taq DNA polymerase incorporates phosphate-labeled nucleotides at an increased rate relative to the Taq DNA polymerase of SEQ ID NO: 766; and
wherein said mutant Taq DNA polymerase comprises one or more phosphate region mutations.

47. The mutant Taq DNA polymerase of claim 46, wherein said one or more additional mutations are selected from the group consisting of a mutation at amino acid positions 589, 617, 645, 691, 673, and 726, and combinations thereof.

48. The mutant Taq DNA polymerase of claim 47, wherein the amino acid at position 617 is isoleucine.

49. The mutant Taq DNA polymerase of claim 47, wherein the amino acid at position 645 is selected from the group consisting of histidine, phenylalanine, lysine and tryptophan.

50. The mutant Taq DNA polymerase of claim 47, wherein the amino acid at position 691 is tyrosine.

51. The mutant Taq DNA polymerase of claim 47, wherein the amino acid at position 693 is glycine.

52. The mutant Taq DNA polymerase of claim 47, wherein the amino acid at position 726 is serine.

53. The mutant Taq DNA polymerase of claim 47, wherein the amino acid at position 589 is aspartic acid and the amino acid at position 645 is histidine.

54. The mutant Taq DNA polymerase of claim 47, wherein said mutant polymerase incorporates phosphate-labeled nucleotides at a rate at least two times faster than the Taq polymerase of SEQ ID NO: 766.

55. The mutant Taq DNA polymerase of claim 47, wherein said mutant polymerase incorporates phosphate-labeled dinucleotides at a rate between five and fifteen times faster than the Taq polymerase of SEQ ID NO: 766.

56. The mutant Taq DNA polymerase of SEQ ID NO: 768, 770, 772, 774, 776, 778, 780, 782 or 784.

57. A purified nucleic acid encoding a mutant Taq DNA polymerase of claim 56.

58. A mutant DNA polymerase selected from the group consisting of the mutant DNA polymerases represented by the even-numbered sequences of SEQ ID NOs: 4-750, 754-764, and 768-784.

59. A mutant DNA polymerase wherein the phosphate region of said mutant DNA polymerase is identical to the phosphate region of a polymerase selected from the group consisting of the mutant DNA polymerases represented by the even-numbered sequences of SEQ ID NOs: 4-750, 754-764, and 768-784.

60. A mutant DNA polymerase selected from the group consisting of the mutant DNA polymerases represented by the even-numbered sequences of SEQ ID NOs: 4-750, wherein said mutant DNA polymerase incorporates phosphate-labeled nucleotides at an increased rate relative to the DNA polymerase of SEQ ID NO: 2.

61. A mutant DNA polymerase selected from the group consisting of the mutant DNA polymerases represented by the even-numbered sequences of SEQ ID NOs: 754-764, wherein said mutant DNA polymerase incorporates phosphate-labeled nucleotides at an increased rate relative to the DNA polymerase of SEQ ID NO: 752.

62. A mutant DNA polymerase selected from the group consisting of the mutant DNA polymerases represented by the even-numbered sequences of SEQ ID NOs: 768-784, wherein said mutant DNA polymerase incorporates phosphate-labeled nucleotides at an increased rate relative to the DNA polymerase of SEQ ID NO: 766.

63. The mutant polymerase of claims 60, 61 or 62, further comprising at least one anchor for attachment to a solid surface.

64. The mutant polymerase of claim 63, wherein said polymerase has at least two anchors.

65. The mutant DNA polymerase of claim 63, wherein said mutant DNA polymerase is used for DNA sequencing and genotyping.

66. The mutant DNA polymerase of claim 65, wherein said DNA sequencing is selected from the group consisting of charge-switch sequencing and electrokinetic sequencing.

67. The mutant DNA polymerase of claim 65, wherein said DNA sequencing is single DNA molecule sequencing.

68. The mutant DNA polymerase of claim 65, wherein said DNA genotyping is single DNA molecule genotyping.

69. A method of DNA sequencing, said method comprising:
(i) providing at least one complex comprising a target nucleic acid, a primer nucleic acid, and a mutant DNA polymerase;
(ii) contacting the complex with a plurality of charged particles comprising at least one type of phosphate-labeled nucleotide triphosphate (NTP) by applying an electric field;
(iii) reversing the electric field to transport unbound charged particles away from the surface; and
(iv) detecting the incorporation of said at least one type of γ-phosphate-labeled NTP into a single molecule of the primer nucleic acid.

70. The method of claim 69, wherein said mutant DNA polymerase is selected from the group consisting of any of the mutants set forth in claims 5, 36, and 46.

71. The method of claim 69, wherein said phosphate-labeled NTP is a γ-phosphate-labeled NTP.

72. The method of claim 71, wherein said γ-phosphate-labeled NTP is further labeled with polyethylene glycol (PEG).
